(19) United States
(12) Patent Application Publication
(10) Pub. No.: US 2025/0041844 A1
(43) Pub. Date: Feb. 6, 2025

(54) Devices and Methods for Transferring Fluid Samples

(71) Applicant: The Trustees of Columbia University in the City of New York, New York, NY (US)

(72) Inventors: Samuel K. SIA, New York, NY (US); Angela TOLWANI, New York, NY (US)

(73) Assignee: The Trustees of Columbia University in the City of New York, New York, NY (US)

(21) Appl. No.: 18/924,043

(22) Filed: Oct. 23, 2024

Related U.S. Application Data
(63) Continuation of application No. PCT/US2023/020003, filed on Apr. 26, 2023.
(60) Provisional application No. 63/334,822, filed on Apr. 26, 2022.

(51) Int. Cl. B01L 3/02

(52) U.S. Cl. CPC B01L 3/0217; B01L 2200/0605; B01L 2300/042; B01L 2300/044

(57) ABSTRACT

Examples of syringe assemblies and sets of components are disclosed for transferring a fluid sample for testing. In some examples, the syringe assembly comprises a housing, a barrel, a plunger reciprocally moveable in the barrel, a narrow tube connected to the barrel, and a metering actuator configured to control movement of the plunger at least in a proximal direction relative to the barrel. The metering actuator is configured to draw a precise microvolume of the fluid sample into a fluid chamber within the syringe assembly. In some examples, while the fluid chamber holds the fluid sample, the distal tip of the narrow tube remains covered, for example inside the housing or a reaction container. The syringe assembly is configured to expel the fluid sample from the fluid chamber into the reaction container.

### RELATED APPLICATIONS

This application is a continuation of International Application No. PCT/US2023/020003, filed Apr. 26, 2023, which claims priority under 35 U.S.C. § 119 (e) to U.S. Provisional Application No. 63/334,822, filed Apr. 26, 2022, and entitled “Devices and Methods for Transferring Fluid Samples,” each of which is incorporated herein by reference in its entirety.

### GOVERNMENT SPONSORSHIP

This invention was made with Government support under Contract No. HL143541 awarded by the National Institutes of Health. The government has certain rights in the invention.

### TECHNICAL FIELD

Devices for transferring fluid samples and related methods are generally described.

### BACKGROUND

The subject matter disclosed herein is directed to overcoming one or more limitations of prior systems and methods for transferring fluid samples, such as from a sample collection container to a reaction container.

Certain prior sample preparation methods are of moderate to high complexity. Certain prior sample preparation methods may only be conducted in CLIA-accredited facilities. Other potential drawbacks or limitations of prior systems and methods include imprecise amounts of samples collected or dispensed, potential exposure of sharp needle tips, high cost, complexity of operation, and/or significant time required.

The subject matter disclosed herein is directed at overcoming one or more of these drawbacks with prior systems and methods.

### SUMMARY

In some embodiments, a syringe assembly for transferring a fluid sample for testing comprises: a housing, a barrel, a plunger reciprocally moveable in the interior of the barrel, a narrow tube (such as a needle or pipette) in fluid communication with the interior of the barrel, and a metering actuator configured to control movement of the plunger at least in a proximal direction relative to the barrel. The barrel, the plunger, and the narrow tube may be configured such that relative movement between the plunger and the barrel expanding a fluid chamber within the syringe assembly causes a fluid sample to be drawn through an opening of the narrow tube into the fluid chamber within the syringe assembly. The housing may be configured to receive a reaction container at the distal end of the housing. The fluid chamber within the syringe assembly may be configured to hold the fluid sample after the fluid sample has been drawn into the fluid chamber and before the fluid sample is expelled from the fluid chamber into the reaction container. The syringe assembly may be configured such that while the fluid chamber holds the fluid sample, the distal tip of the narrow tube remains covered inside the housing proximal to the distal end of the housing. The barrel, the plunger, and the narrow tube may be configured such that relative movement between the plunger and the barrel contracting the fluid chamber causes the fluid sample to be expelled from the fluid chamber through the opening of the narrow tube into the reaction container.

In some embodiments, a set of components usable together for transferring a fluid sample for testing comprises a syringe assembly and a collection container assembly and/or a reaction container assembly. The syringe assembly may comprise components as described above.

In some embodiments, a method of transferring a fluid sample for testing comprises: (i) using a syringe assembly to draw a fluid sample, the syringe assembly comprising: a housing, a barrel, a plunger reciprocally moveable in the interior of the barrel in a proximal direction and in a distal direction, a narrow tube in fluid communication with the interior of the barrel, and a metering actuator configured to control movement of the plunger at least in a proximal direction relative to the barrel; wherein using the syringe assembly to draw the fluid sample comprises creating relative movement between the plunger and the barrel expanding a fluid chamber within the syringe assembly thereby causing the fluid sample to be drawn through the opening of the narrow tube into the fluid chamber within the syringe assembly; (ii) causing a reaction container to be received at the distal end of the housing; (iii) holding the fluid sample in the fluid chamber after the fluid sample has been drawn into the fluid chamber and before the fluid sample is expelled from the fluid chamber into the reaction container, wherein while the fluid chamber is holding the fluid sample, the distal tip of the narrow tube remains covered inside the housing proximal to the distal end of the housing; and (iv) creating relative movement between the plunger and the barrel contracting the fluid chamber thereby causing the fluid sample to be expelled from the fluid chamber through the opening of the narrow tube into the reaction container. The method may further comprise, after expelling the fluid sample from the barrel into the reaction container, exposing the reaction container to one or more polymerase chain reaction processing steps while the reaction container remains secured to the syringe assembly.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 illustrates a first embodiment of a set of components, including a cartridge or syringe assembly for transferring a fluid sample for testing, along with a collection container assembly and a reaction container assembly, according to one set of embodiments;

FIG. 2 shows an exploded view of the syringe assembly of FIG. 1, according to one set of embodiments;

FIG. 3 shows a portion of the syringe assembly of FIG. 1, illustrating different operating positions, according to one set of embodiments;

FIG. 4 shows the reaction container assembly of FIG. 1, according to one set of embodiments;

FIGS. 5A and 5B show the syringe assembly of FIG. 1 in different operating positions prior to receiving a fluid sample, according to one set of embodiments;

FIGS. 6A through 6C show stages in drawing a fluid sample from a collection container into the syringe assembly of FIG. 1, according to one set of embodiments;

FIGS. 7A through 7C show stages in expelling the fluid sample from the syringe assembly of FIG. 1 into a reaction container, according to one set of embodiments;

FIGS. 8A and 8B show further stages in use of the syringe assembly of FIG. 1, according to one set of embodiments;

FIG. 9 illustrates an exploded view of a second embodiment of a set of components, including a cartridge or syringe assembly for transferring a fluid sample for testing, along with a reaction container assembly, according to one set of embodiments;

FIGS. 10A through 10C show stages in drawing a fluid sample into the fluid chamber of the syringe assembly of FIG. 9, according to one set of embodiments;

FIGS. 11A and 11B show stages in expelling the fluid sample from the fluid chamber of the syringe assembly of FIG. 9 into a reaction container, according to one set of embodiments;

FIGS. 12A and 12B illustrate side and cross-sectional views, respectively, of a third embodiment of a cartridge or syringe assembly for transferring a fluid sample for testing, suitable for use in a third embodiment of a set of components further including a collection container assembly and a reaction container assembly, according to one set of embodiments;

FIG. 13 illustrates a fourth embodiment of a set of components, including a cartridge or syringe assembly for transferring a fluid sample for testing, along with a collection container assembly and a reaction container assembly, according to one set of embodiments;

FIGS. 14A and 14B show side and cross-sectional views, respectively, of the syringe assembly of FIG. 13 prior to receiving a fluid sample, according to one set of embodiments;

FIGS. 15A and 15B show side and cross-sectional views, respectively, of the syringe assembly of FIG. 13 with a collection container assembly received within the syringe assembly, according to one set of embodiments;

FIGS. 16A and 16B show side and cross-sectional views, respectively, of the syringe assembly of FIG. 13 with the fluid sample drawn from the collection container into the fluid chamber of the syringe assembly, according to one set of embodiments;

FIGS. 17A and 17B show side and cross-sectional views, respectively, of the syringe assembly of FIG. 13 with the fluid sample drawn into the fluid chamber of the syringe assembly and with the collection container assembly removed, according to one set of embodiments;

FIGS. 18A and 18B show side and cross-sectional views, respectively, of the syringe assembly of FIG. 13 with a reaction container assembly received within the syringe assembly, according to one set of embodiments;

FIGS. 18C and 18D show side and cross-sectional views, respectively, of the syringe assembly of FIG. 13 with the fluid sample expelled into the reaction container, according to one set of embodiments;

FIGS. 19A and 19B show side and cross-sectional views, respectively, of the syringe assembly of FIG. 13 with the fluid sample expelled into the reaction container and with the reaction container cover removed, according to one set of embodiments;

FIGS. 20A and 20B show side and cross-sectional views, respectively, of the syringe assembly of FIG. 13 with the fluid sample expelled into the reaction container and with the housing cover removed, according to one set of embodiments;

FIG. 21 illustrates the syringe assembly of FIG. 13 with the attached reaction container being transferred to a testing apparatus, according to one set of embodiments;

FIGS. 22A and 22B show side and cross-sectional views, respectively, of a fifth embodiment of a cartridge or syringe assembly for transferring a fluid sample for testing, along with a collection container assembly received within the syringe assembly, according to one set of embodiments;

FIGS. 23A and 23B show side and cross-sectional views, respectively, of the syringe assembly of FIGS. 22A and 22B with the fluid sample drawn from the collection container into the fluid chamber of the syringe assembly, according to one set of embodiments;

FIGS. 24A and 24B show side and cross-sectional views, respectively, of the syringe assembly of FIGS. 22A and 22B with the fluid sample drawn into the fluid chamber of the syringe assembly, with the collection container assembly removed, and with a reaction container assembly received within the syringe assembly, according to one set of embodiments;

FIG. 25 shows a cross-sectional view of the syringe assembly of FIGS. 22A and 22B with the fluid sample expelled into the reaction container, according to one set of embodiments; and

FIG. 26 shows a side view of the syringe assembly of FIGS. 22A and 22B with the fluid sample expelled into the reaction container, with the reaction container cover removed, and with the housing cover removed, according to one set of embodiments.

### DETAILED DESCRIPTION

The following description of illustrated embodiments serves to disclose examples. The scope of the subject matter disclosed herein includes numerous variations incorporating one or more of the features described herein.

Embodiments as disclosed herein involve transferring a fluid sample, such as a sample of saliva or blood, for testing. Certain embodiments as disclosed herein involve collecting a fluid sample from a collection container, such as a collection vial or collection tube, and/or dispensing the fluid sample into a reaction container, such as a reaction vial or reaction tube. The collection container and/or reaction container may be covered by a pierceable cover, such as a stopper, septum, or cap, which may be made of any suitable material, such as elastomeric materials, metallic or other films, or plastic. The pierceable cover may be pierceable by a narrow tube, such as a needle or pipette tip, which may be made of any suitable material, such as metal or plastic. As described in more detail further below, the embodiments described herein may eliminate some of the challenges and risks associated with certain existing transferring or dispending techniques. For example, certain existing transfer pipettes are fabricated from one, continuous sheet of plastic, including the bulb of the pipette tip and, hence, lack precision in receiving and transferring a precise amount of sample. Moreover, these transfer pipette tips lack precision when dispensing an obtained sample and may result in sample leakage or loss during transfer, and may also result in too much or too little sample being dispended. Certain existing micropipettes attempt to improve upon the lack of precision of transfer pipettes by including more precise control over the volume of sample obtained and/or dispended; however, these micropipettes require more training for the user than existing transfer pipettes. These micropipettes also have a risk of malfunctioning if they are improperly calibrated for a particular volume and may cease functioning properly altogether. Further still, these existing transferring pipettes are generally not suitable for reactions (e.g., cannot hold and/or placed in direct physical connection with a reaction container).

It has been recognized and appreciated by the present disclosure that precise volumes of sample may be drawn and/or transferred to one container (e.g., a container comprising a biological sample) to another container (e.g., a reaction container) in a fewer numbers of steps that reduces potential user error, and/or without the use of existing transfer apparatuses (e.g., transfer pipettes, micropipettes). Advantageously, these volumes may be transferred without the need of pipette bulbs or the like, and can be handled relatively simply without the need for extensive training or the risk of over calibration by the user. In some cases the syringe assembly includes a tube (e.g., a narrow tube) and a plunger that can be controlled by the operator (e.g., via a metering actuator) such that the volume of sample received by the tube is of a fixed volume, which facilitates ease of use, relative to existing fluid transferring devices.

To facilitate the transfer of a sample (e.g., a saliva sample, a blood sample) to a reaction container, some embodiments of the syringe assembly described herein include a housing that is configured to receive a reaction container. In some embodiments, the syringe assembly has a shape and/or configuration that is complementary to the shape and/or configuration of a reaction container assembly and/or a reaction container holder. The reaction container may, for instance, be configured to attach (e.g., reversibly attach, irreversibly attach) directly to a portion of the syringe assembly. For example, in some embodiments, a reaction container assembly includes a reaction container holder, and this reaction container holder includes one or more projections, and these projections may complement another portion of a syringe assembly (e.g., a cap of the syringe assembly) so that the reaction container attaches (e.g., reversibly attaches) directly to the remainder of the syringe assembly. Of course, other configurations are possible that facilitate attachment of the reaction container to the syringe assembly. Details regarding attachment of the reaction container to the syringe assembly are described below.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

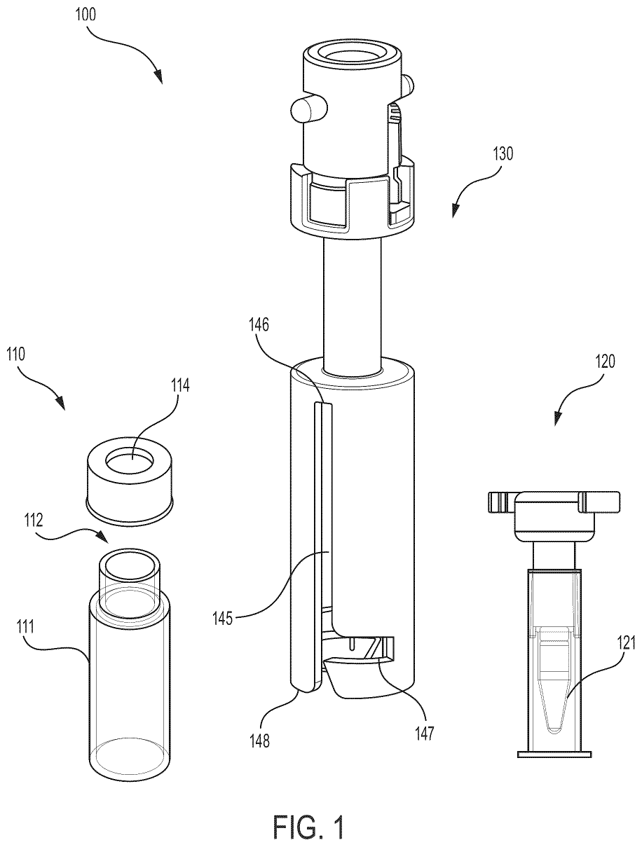

FIGS. 1 through 8B illustrate a first embodiment of a set 100 of components including a cartridge or syringe assembly 130 for transferring a fluid sample for testing. The set 100 of components includes the syringe assembly 130, a collection container assembly 110, and a reaction container assembly 120.

The collection container assembly 110 includes collection container 111 in which a fluid sample, such as a sample of saliva or blood (e.g., from a patient), may be collected. The collection container has an opening 112 at its top end. The opening 112 is covered by a pierceable cover 114 that is pierceable by a narrow tube such as a needle or pipette tip to access the contents of the collection container 111.

As discussed in more detail below and elsewhere herein, various embodiments include a narrow tube and the narrow tube (e.g., a fluid chamber of the narrow tube) may have dimensions suitable for receiving, holding, and/or transferring a particular amount of sample. For example, in some embodiments, the narrow tube has an average diameter of greater than or equal to 1 μm, greater than or equal to 10 μm, greater than or equal to 20 μm, greater than or equal to 50 μm, greater than or equal to 75 μm, greater than or equal to 100 μm, greater than or equal to 200 μm, greater than or equal to 250 μm, greater than or equal to 300 μm, greater than or equal to 500 μm, greater than or equal to 700 μm, greater than or equal to 750 μm, greater than or equal to 1 mm, greater than or equal to 2 mm, greater than or equal to 3 mm, or greater than or equal to 5 mm. In some embodiments, the narrow tube has an average diameter of less than or equal to 5 mm, less than or equal to 3 mm, less than or equal to 2 mm, less than or equal to 1 mm, less than or equal to 750 μm, less than or equal to 700 μm, less than or equal to 500 μm, less than or equal to 300 μm, less than or equal to 250 μm, less than or equal to 200 μm, less than or equal to 100 μm, less than or equal to 50 μm, less than or equal to 20 μm, less than or equal to 10 μm, or less than or equal to 1 μm. Combinations of the above-referenced ranges are also possible (e.g., greater than or equal to 1 μm and less than or equal to 5 mm). Other ranges are possible.

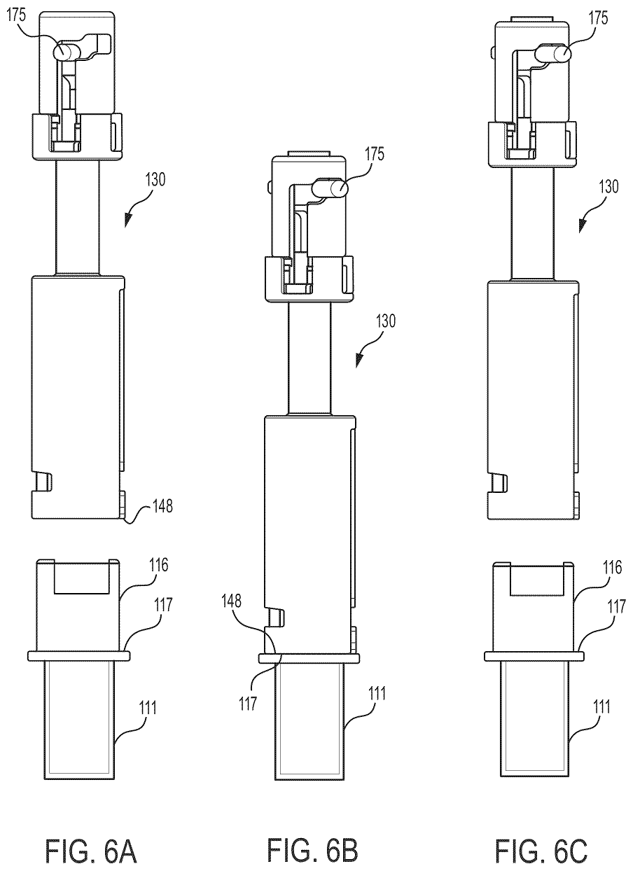

As shown in FIGS. 6A-6C, the collection container assembly 110 may also include a cap 116 that fits over the top of the collection container 111. The cap 116 may have a flange 117 that acts as a stop when inserting the collection container assembly 110 in the syringe assembly 130, as described below.

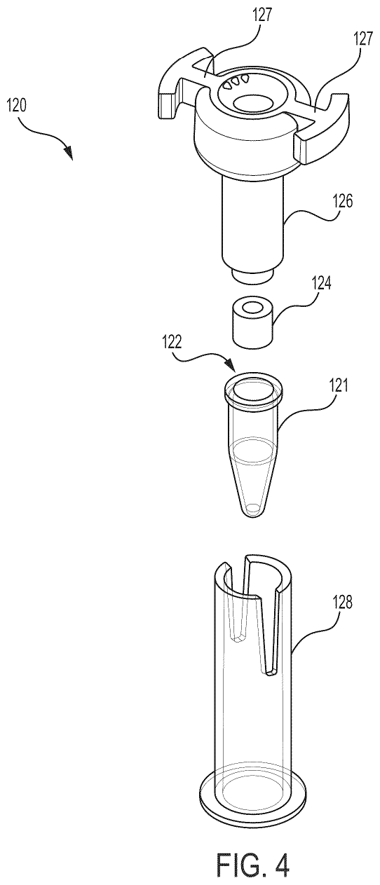

As can be seen in FIG. 4, the reaction container assembly 120 comprises a reaction container 121 having an opening 122 at its top end. The opening 122 is covered by a pierceable cover 124 that is pierceable by a narrow tube such as a needle or pipette tip to dispense a sample into the reaction container 121. The reaction container assembly 120 also comprises a reaction container holder 126 for holding the reaction container 121. The reaction container holder 126 has one or more projections 127. The reaction container assembly 120 further includes a protective reaction container cap or cover 128.

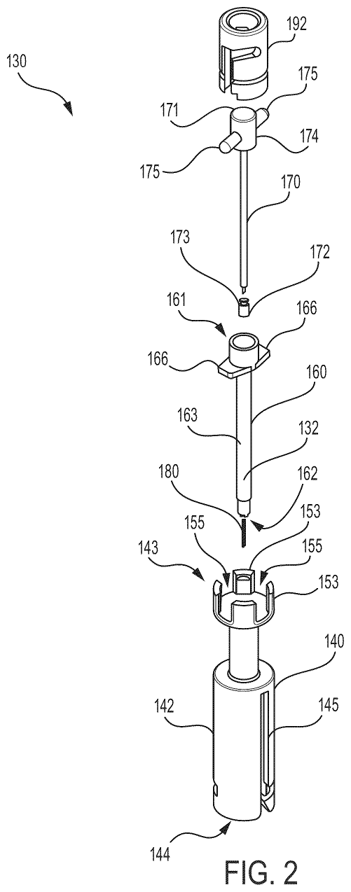

FIG. 2 shows an exploded view of the syringe assembly 130. The syringe assembly 130 includes a housing 140 having a side wall 142, a proximal end 143, and a distal end 144. The side wall 142 of the housing 140 has a slot or track 145 in it for receiving the reaction container assembly 120, as described below. As can be seen in FIG. 1, the track 145 has a proximal reaction container stop 146 and a distal reaction container stop 147 for different positionings of the reaction container assembly 120. The distal end 144 of the housing also serves as a collection container stop 148, as described below.

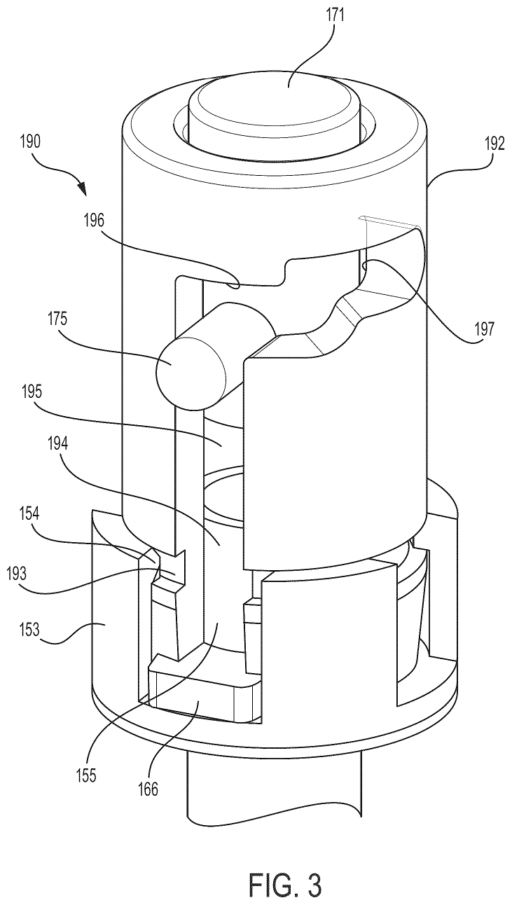

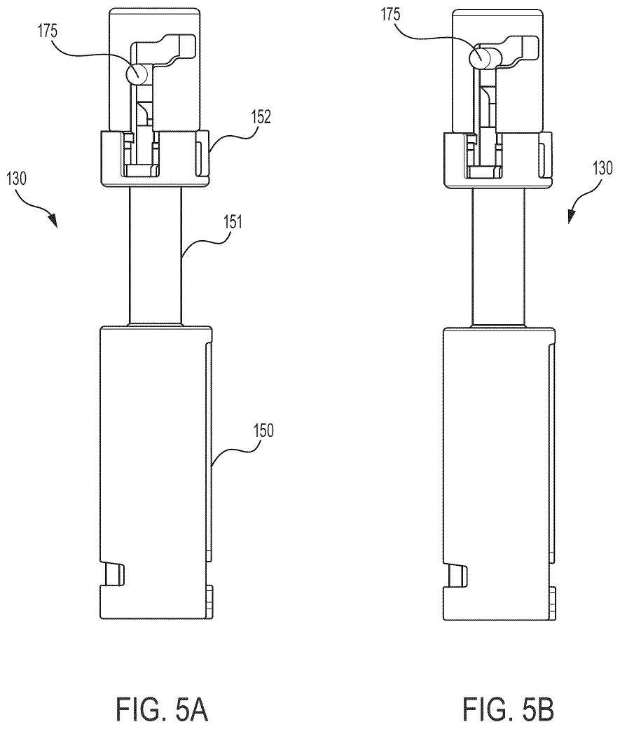

In this example embodiment, and as can be seen in FIG. 5A, the housing 140 can be considered as having three parts, a lower part 150, a middle part 151, and an upper part 152. The lower part 150 of the housing 140 receives the collection container assembly 110 for withdrawing a fluid sample from the collection container 111 and the reaction container assembly 120 for expelling the fluid sample into the reaction container 121. The middle part 151 of the housing 140 forms a passageway between the upper part 152 and the lower part 150. The upper part 152 of the housing 140 includes an engagement mechanism in the form of resilient tabs 153 with projections 154 at their ends, as can be seen in FIG. 3. The resilient tabs 153 are spaced apart, leaving spaces 155 between resilient tabs 153.

As shown in FIG. 2, the syringe assembly 130 also includes a syringe barrel 160. The syringe barrel 160 has a proximal end 161, a distal end 162, and an interior 163. The proximal end 161 of the barrel 160 has one or more tabs 166. When the syringe assembly 130 is assembled, at least the distal end 162 of the barrel 160 is located inside the housing 140. When the syringe assembly 130 is assembled, the tabs 166 of the barrel 160 are positioned in the spaces 155 between the resilient tabs 153, as can be seen in FIG. 3.

The syringe assembly 130 also includes a plunger 170. The plunger 170 has a proximal end 171 and a distal end 172. The plunger 170 has a stopper 173 at its distal end 172. The plunger 170 is reciprocally moveable in the interior 163 of the barrel 160 in a proximal direction and in a distal direction. The plunger 170 has an enlarged part 174 at its proximal end 171 and one or more stems 175 that project radially outwardly.

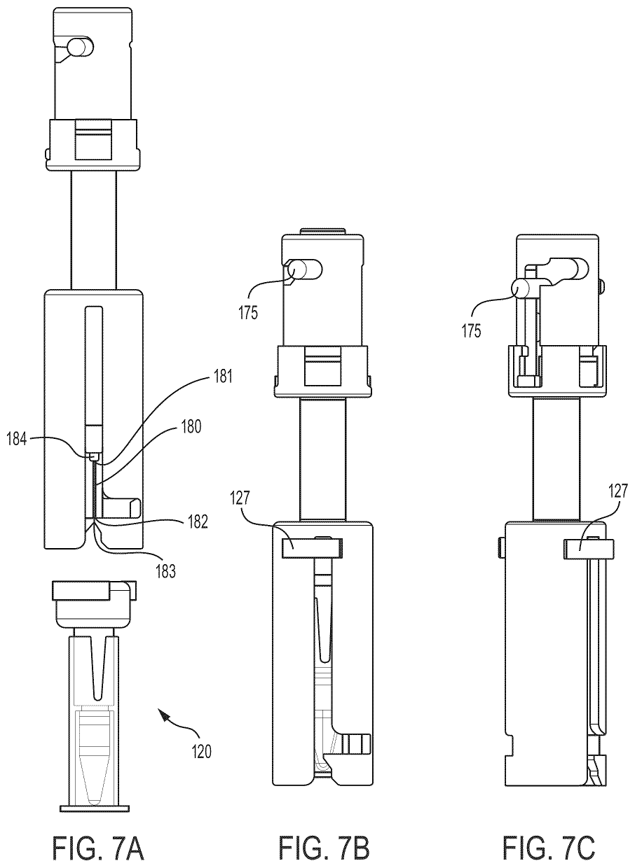

The syringe assembly 130 further includes a narrow tube 180, such as a needle or pipette tip. As can be seen in FIG. 7A, the narrow tube 180 has a proximal end 181 and a distal tip 182. The proximal end 181 of the narrow tube 180 is connected by a hub 184 to the distal end 162 of the barrel 160. The narrow tube 180 has an interior, and the proximal end of the narrow tube interior is in fluid communication with a distal end of the interior 163 of the barrel 160. The plunger 170 and the barrel 160, and in some embodiments also the narrow tube 180, define a fluid chamber 132 within the interior 163 of the barrel 160 and/or the interior of the narrow tube 180. The distal tip 182 of the narrow tube 180 has an opening 183 such that a fluid sample can be drawn into the opening 183 and into the fluid chamber 132, and such that a fluid sample can be expelled from the fluid chamber 132 out of the opening 183.

The syringe assembly 130 also includes a cap 192. As can be seen in FIG. 3, the cap 192 has a groove 193 that extends around its circumference for receiving the projections 154 of the upper part 152 of the housing 140. The cap also has one or more slots 194 for receiving one or more stems 175 of the plunger 170.

In the illustrated example, the stem(s) 175 and the cap 192 together form a metering actuator 190 that is configured to control movement of the plunger 170 relative to the barrel 160 in both proximal and distal directions. As shown in FIG. 3, the slot 194 has a vertical portion 195, a first stop 196, and a second stop 197 for positioning the stem 175 and consequently the plunger 170 in the desired position.

The barrel 160, the plunger 170, and the narrow tube 180 are configured such that relative movement between the plunger 170 and the barrel 160 that expands the fluid chamber 132 causes a fluid sample to be drawn through the opening 183 of narrow tube 180 into the fluid chamber 132. Similarly, the barrel 160, the plunger 170, and the narrow tube 180 are configured such that relative movement between the plunger 170 and the barrel 160 that contracts the fluid chamber 132 causes the fluid sample to be expelled from the fluid chamber 132 through the opening 183 of narrow tube 180.

FIGS. 5A through 8B show various stages in transferring a fluid sample for testing. FIG. 5A shows the syringe assembly 130 in a neutral position, prior to receiving the sample. The stem 175 is in the distal position, identified as position 3 in FIG. 3. To get the syringe assembly 130 into position to draw the sample, the operator moves the stem 175 from position 3 in FIG. 3 to the first stop 196 at position 1 in FIG. 3, reaching the ready position shown in FIG. 5B.

FIGS. 6A through 6C show stages in drawing a fluid sample from the collection container 111. A fluid sample has been collected from a patient and is in the collection container 111, optionally along with a buffer for certain applications. The operator inserts the collection container assembly 110 into the opening at the distal end 144 of the housing 140 until the collection container stop 117 of the collection container assembly 110 abuts the collection container stop 148 of the housing 140, as shown in FIG. 6B. This limits further motion of the collection container assembly 110 into the housing 140. In this position, the narrow tube 180 is in the collection container 111 in contact with the fluid sample. With the collection container assembly 110 in place, the operator then moves the stem 175 from position 1 in FIG. 3 to the second stop 197 at position 2 in FIG. 3. This results in moving the plunger 170 proximally relative to the barrel 160 by a predetermined amount, defined by the longitudinal distance between position 1 and position 2. This results in a precise amount of the fluid sample being drawn through the opening 183 of the narrow tube 180 into the fluid chamber 132. The relative movement between the plunger 170 and the barrel 160 expands the fluid chamber 132, thereby causing the fluid sample to be drawn through the opening 183 of narrow tube 180 into the fluid chamber 132. Once the fluid sample is drawn into the fluid chamber 132, the operator can remove the collection container assembly 110, as shown in FIG. 6C. At this stage—after the fluid sample has been drawn into the fluid chamber 132 and before the fluid sample is expelled from the fluid chamber 132 into the reaction container—the fluid sample is held in the fluid chamber 132, while the distal tip 182 of the narrow tube 180 remains covered inside the housing 140 proximal to the distal end 144 of the housing 140.

FIGS. 7A through 7C show stages in expelling the fluid sample from the fluid chamber 132 into the reaction container 121. The reaction container 121 may have desired reagents in it pertinent to a particular application. The reaction container assembly 120 is received at the distal end 144 of the housing 140 by the operator inserting the reaction container assembly 120 into the distal end 144 of the housing 140. The projections 127 of the reaction container holder 126 fit into the tracks 145. The operator advances the reaction container assembly 120 into the housing 140 until the projections 127 reach the proximal reaction container stops 146. This position is shown in FIG. 7B. In this position, the narrow tube 180 is in the reaction container 121. In this position, the operator then moves the stem 175 from position 2 in FIG. 3 back to position 3 in FIG. 3. This moves the plunger 170 fully distally within the barrel 160. The relative movement between the plunger 170 and the barrel 160 in this direction contracts the fluid chamber 132 within the interior 163 of the barrel 160, thereby causing the fluid sample to be expelled from the fluid chamber 132 through the opening 183 of the narrow tube 180 into the reaction container 121. The fully advanced position of the stem 175 is shown in FIG. 7C.

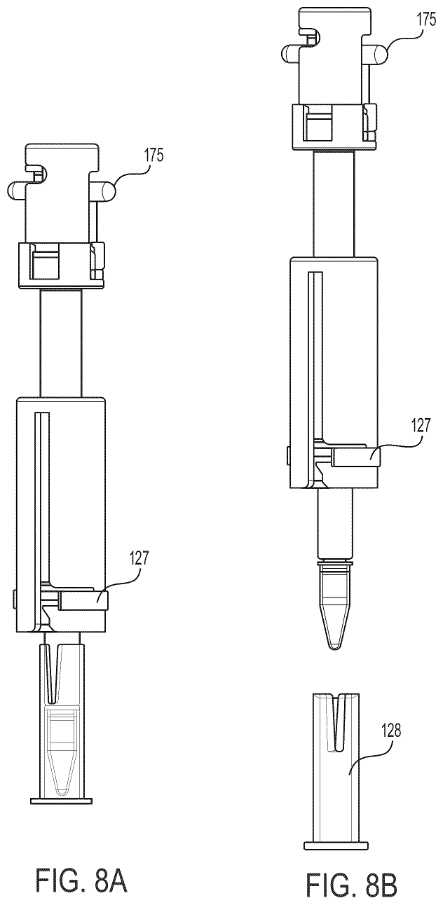

FIGS. 8A and 8B show further stages in use of the syringe assembly 130. After the fluid sample is expelled into the reaction container 121, the operator moves the reaction container assembly 120 from its proximal position shown in FIG. 7C to its distal position shown in FIG. 8A. The projections 127 of the reaction container holder 126 are moved distally in the tracks 145 until reaching the distal reaction container stop 147. The reaction container assembly 120 may be rotated within the circumferential extension of the tracks 145 to the distal reaction container stop 147. In this position, the operator can remove the reaction container cover 128 from the reaction container assembly 120, as shown in FIG. 8B. In this condition, the cartridge or syringe assembly 130 holds the reaction container 121 with the fluid sample in it. By handling the syringe assembly 130, the operator can transfer the reaction container 121 to a testing apparatus, such as a polymerase chain reaction machine, to expose the reaction container 121 to one or more processing steps, such as polymerase chain reaction processing steps, while the reaction container 121 remains secured to the syringe assembly 130.

In the embodiment of FIGS. 1 through 8B, the distal tip of the narrow tube 180 is configured to remain at all times located inside the housing 140 proximal to the distal end 144 of the housing 140. Also, during part of the operation, the distal tip of the narrow tube 180 is located inside the collection container 111 or located inside the reaction container 121.

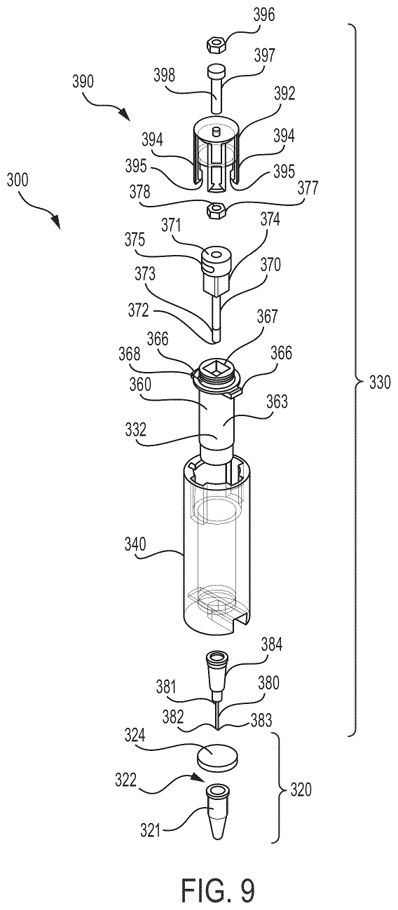

FIGS. 9 through 11B illustrate a second embodiment of a set 300 of components including a cartridge or syringe assembly 330 for transferring a fluid sample for testing. In this embodiment, the set 300 of components includes the syringe assembly 330 and a reaction container assembly 320.

In this embodiment, the reaction container assembly 320 comprises a reaction container 321 having an opening 322 at its top end. The opening 322 is covered by a pierceable cover 324 that is pierceable by a narrow tube such as a needle or pipette tip to dispense a sample into the reaction container 321. The pierceable cover 324 has a projecting edge 327 that facilitates securing the reaction container assembly 320 to the syringe assembly 330.

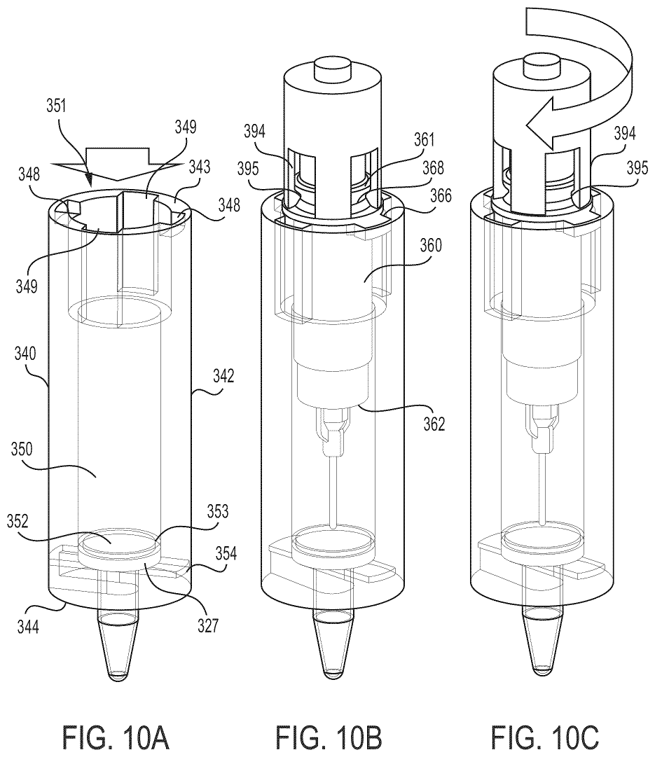

FIG. 9 shows an exploded view of the syringe assembly 330 and the reaction container assembly 320. The syringe assembly 330 includes a housing 340 that, as can be seen in FIG. 10A, has a side wall 342, a proximal end 343, and a distal end 344. The distal end 344 of the housing 340 has a slot or track 354 in it for receiving the reaction container assembly 320, by the projecting edge 327 of the pierceable cover 324 fitting within the slot 354. The proximal end 343 of the housing 340 has a set of short first slots 348 and a set of longer second slots 349 for receiving tabs 366 of the barrel 360 in different operating positions.

In this example embodiment, the interior of the housing 340 is a sample chamber 350 that functions as the collection container. The sample chamber 350 has a top opening 351 and a bottom 352 that is covered by a pierceable cover 353, such as a stopper or septum. The patient or operator can directly dispense the fluid sample into the sample chamber 350, or the fluid sample can be collected in a separate sample collection container and transferred to the sample chamber 350.

As shown in FIG. 9, the syringe assembly 330 also includes a syringe barrel 360. As can be seen in FIGS. 9 and 10B, the syringe barrel 360 has a proximal end 361, a distal end 362, and an interior 363. The proximal end 361 of the barrel 360 has one or more tabs 366. When the syringe assembly 330 is assembled, at least the distal end 362 of the barrel 360 is located inside the housing 340. In this embodiment, the barrel 360 further includes a portion 367 at which the interior has a non-circular cross-section. The barrel 360 also has a 368 groove on an outer surface of barrel 360 extending around the circumference of the barrel 360.

The syringe assembly 330 also includes a plunger 370. The plunger 370 has a proximal end 371 and a distal end 372. The plunger 370 has a stopper 373 at its distal end 372. The plunger 370 is reciprocally moveable in the interior 363 of the barrel 360 in a proximal direction and in a distal direction. The plunger 370 also has a portion 374 at which the exterior has a non-circular cross-section. The portion 374 of the plunger 370 is configured to fit within the corresponding portion 367 of the barrel 360 such that the plunger 370 can reciprocate within the barrel 360 in a longitudinal direction but is restrained by the mating shapes from rotating within the barrel 360. At its proximal end 371, the plunger 370 has a slot 375 for receiving a nut 377 in a manner such that the nut 377 is restrained from rotating relative to the plunger 370. For example, the nut 377 may be hexagonal or have another non-circular shape fitting within a slot 375 shaped to receive the nut 377 and inhibit it from rotating. The nut 377 has an internally-threaded surface 378. In an alternative embodiment, an internal surface of the plunger 370 may be internally threaded such that a separate nut 377 is not needed.

The syringe assembly 330 further includes a narrow tube 380, such as a needle or pipette tip. The narrow tube 380 has a proximal end 381 and a distal tip 382. The proximal end 381 of the narrow tube 380 is connected by a hub 384 to the distal end 362 of the barrel 360. The narrow tube 380 has an interior, and the proximal end of the narrow tube interior is in fluid communication with a distal end of the interior 363 of the barrel 360. The plunger 370 and the barrel 360, and in some embodiments also the narrow tube 380, define a fluid chamber 332 within the interior 363 of the barrel 360 and/or the interior of the narrow tube 380. The distal tip 382 of the narrow tube 380 has an opening 383 such that a fluid sample can be drawn into the opening 383 and into the fluid chamber 332, and such that a fluid sample can be expelled from the fluid chamber 332 out of the opening 383.

The syringe assembly 330 also includes a cap 392. The cap 392 has a plurality of resilient tabs 394 which project downwardly. Each of the tabs 394 has an inwardly-facing projection 395 at its end. When assembled, the projections 395 engage the groove 368 of the barrel 360, allowing the cap 392 to be rotated relative to the barrel 360 while restraining separation of the cap 392 from the barrel 360. A screw 397 is attached to the cap 392, extending through a hole in the top of the cap 392. The screw 397 has an externally-threaded surface 398. The head of the screw 397 is on the top side of the cap 392, while a nut 396 on the screw 397 is on the underside of the cap 392, securing the screw 397 to the cap 392. In an alternative embodiment, the cap 392 has an externally-threaded post such that a separate screw 397 is not needed.

The cap 392, screw 397, and nut 377 together form a metering actuator 390 that is configured to control movement of the plunger 370 relative to the barrel 360 in both proximal and distal directions. Rotation of the cap 392 causes rotation of the screw 397, because the screw is secured to the cap 392. The externally-threaded surface 398 of the screw 397 is in mating engagement with the internally-threaded surface 378 of the nut 377. The nut 377 is inhibited from rotation due to the engagement between the nut 377 and the plunger 370 and the engagement between the plunger 370 and the barrel 360. Thus, when the cap 392 is rotated causing the screw 397 to rotate, the threaded engagement with the nut 377 causes the nut 377 to move longitudinally with respect to the screw 397. This causes the plunger 370 to move longitudinally within the barrel 360.

The barrel 360, the plunger 370, and the narrow tube 380 are configured such that relative movement between the plunger 370 and the barrel 360 that expands the fluid chamber 332 causes a fluid sample to be drawn through the opening 383 of narrow tube 380 into the fluid chamber 332. Similarly, the barrel 360, the plunger 370, and the narrow tube 380 are configured such that relative movement between the plunger 370 and the barrel 360 that contracts the fluid chamber 332 causes the fluid sample to be expelled from the fluid chamber 332 through the opening 383 of the narrow tube 380.

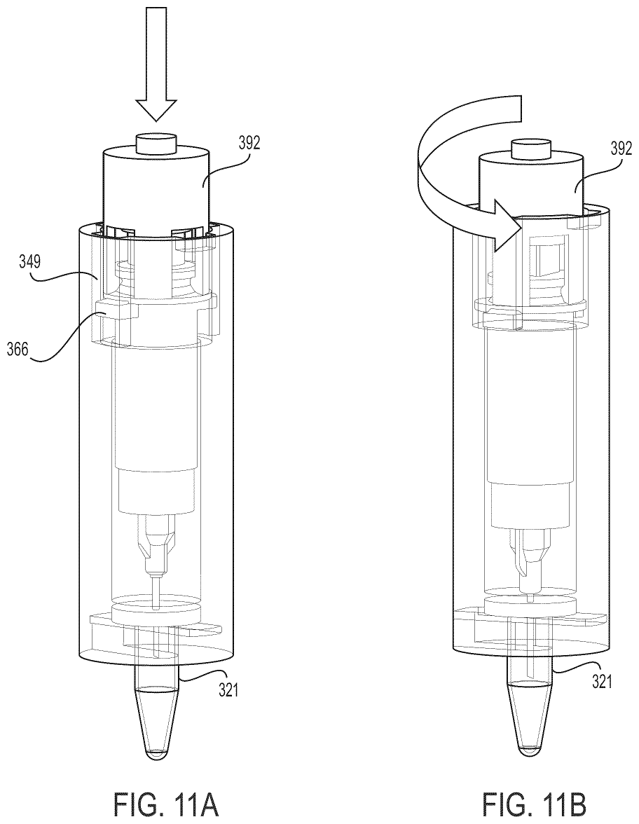

FIGS. 10A through 11B show various stages in transferring a fluid sample for testing. FIG. 10A shows the housing 340 of the syringe assembly 350 with a fluid sample received in the sample chamber 350. As shown in FIG. 10B, the remaining components of the syringe assembly 330, including the barrel 360, the plunger 370, the narrow tube 380, and the cap 392, which are all assembled together, are placed into the top opening 351 of the housing 340 with the assembly in a first position in which the tabs 366 of the barrel 360 are positioned in the short first slots 348 in the housing 340. The positioning of the tabs 366 in the first slots 348 sets the depth of insertion of the narrow tube 380 within the housing 340 and also inhibits rotation of the barrel 360 with respect to the housing 340. In this position, the narrow tube 380 is within the sample chamber 350.

From the position shown in FIG. 10B, the operator then rotates the cap 392 in a first direction, as shown in FIG. 10C. This action draws the fluid sample from the sample chamber 350 through the opening 383 of the narrow tube 380 into the fluid chamber 332. The cap 392 is rotated until the proximal end 371 of the plunger 370 abuts a stop within the cap 392, inhibiting further rotation of the cap 392. This full rotation of the cap 392 to the stop position results in moving the plunger 370 proximally relative to the barrel 360 by a predetermined amount. This results in a precise amount of the fluid sample being drawn into the fluid chamber 332. The relative movement between the plunger 370 and the barrel 360 expands the fluid chamber 332, thereby causing the fluid sample to be drawn through the opening 383 of narrow tube 380 into the fluid chamber 332. As shown in FIG. 10C, at this stage—after the fluid sample has been drawn into the fluid chamber 332 and before the fluid sample is expelled from the fluid chamber 332 into the reaction container—the fluid sample is held in the fluid chamber 332, while the distal tip 382 of the narrow tube 380 remains covered inside the housing 340 proximal to the distal end 344 of the housing 340.

FIGS. 11A and 11B show stages in expelling the fluid sample from the fluid chamber 332 into the reaction container 321. The reaction container 321 may have reagents in it for a desired application. The reaction container assembly 320 is received at the distal end 344 of the housing 340 by the operator inserting the reaction container assembly 320 into the distal end 344 of the housing 340. The projecting edge 327 of the pierceable cover 324 fits into the slot 354.

After the fluid sample is drawn into the fluid chamber 332, as in FIG. 10C, the operator switches the operating position between the housing 340 and the remaining components of the syringe assembly 330. The operator lifts the remaining components of the syringe assembly 330 to withdraw the tabs 366 from the first slots 348. The operator turns the remaining components of the syringe assembly 330 to position the tabs 366 above the second slots 349, and moves these assembled components distally, as shown in FIG. 11A, until the tabs 366 reach the distal ends of the second slots 349. The second slots 349 are longer than the first slots 348. The positioning of the tabs 366 in the second slots 348 sets a different depth of insertion of the narrow tube 380, wherein the narrow tube 380 pierces the cover 353 at the bottom of the sample chamber 350 and the pierceable cover 324 at the top of the reaction container 321. The positioning of the tabs 366 in the second slots 348 also inhibits rotation of the barrel 360 with respect to the housing 340.

In this position, as shown in FIG. 11B, the operator then rotates the cap 392 in a second direction, opposite to the first direction. This action expels the fluid sample from the fluid chamber 332 through the opening 383 of the narrow tube 380 into the reaction container 321. The cap 392 is rotated fully until it is inhibited from further rotation. This moves the plunger 370 fully distally within the barrel 360. The relative movement between the plunger 370 and the barrel 360 in this direction contracts the fluid chamber 332, thereby causing the fluid sample to be expelled from the fluid chamber 332 through the opening 383 of the narrow tube 380 into the reaction container 321, as shown in FIG. 11B.

After the fluid sample is expelled into the reaction container 321, the operation can transfer the reaction container 321 to a testing apparatus, such as a polymerase chain reaction machine, as described above, to expose the reaction container 321 to one or more processing steps, such as polymerase chain reaction processing steps, while the reaction container 321 remains secured to the syringe assembly 330.

In the embodiment of FIGS. 9 through 11B, the distal tip of the narrow tube 380 is configured to remain at all times located inside the housing 340 proximal to the distal end 344 of the housing 340 or located inside the reaction container 321. For example, in FIG. 11B, the distal tip of the narrow tube 380 is located beyond the distal end 344 of the housing 340 but is located inside the reaction container 321 and is thereby protected from exposure to a person handling the device.

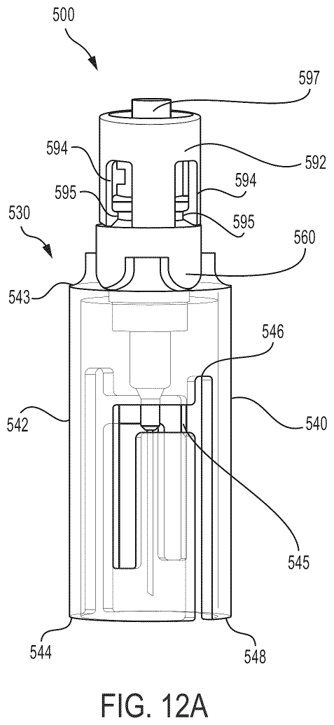

FIGS. 12A and 12B illustrate a third embodiment of a set 500 of components including a cartridge or syringe assembly 530 for transferring a fluid sample for testing. The set 500 of components includes the syringe assembly 530 and may also include a collection container assembly similar to the collection container assembly 110 and/or a reaction container assembly similar to the reaction container assembly 120.

The syringe assembly 530 includes a housing 540 having a side wall 542, a proximal end 543, and a distal end 544. The side wall 542 of the housing 540 has a slot or track 545 in it for receiving the reaction container assembly 120, similar to the track 145 above receiving the reaction container assembly 120. The track 545 has a proximal reaction container stop 546 and a distal reaction container stop 547 for different positionings of the reaction container assembly 120. The distal end 544 of the housing also serves as a collection container stop 548.

The syringe assembly 530 also includes a syringe barrel 560. The syringe barrel 560 has a proximal end 561, a distal end 562, and an interior 563. When the syringe assembly 530 is assembled, at least the distal end 562 of the barrel 560 is located inside the housing 540. In this embodiment, the barrel 560 further includes a portion 567 at which the interior has a non-circular cross-section. The barrel 560 also has a 568 groove on an outer surface of barrel 560 extending around the circumference of the barrel 560.

The syringe assembly 530 also includes a plunger 570. The plunger 570 has a proximal end 571 and a distal end 572. The plunger 570 has a stopper 573 at its distal end 572. The plunger 570 is reciprocally moveable in the interior 563 of the barrel 560 in a proximal direction and in a distal direction. The plunger 570 also has a portion 574 at which the exterior has a non-circular cross-section. The portion 574 of the plunger 570 is configured to fit within the corresponding portion 567 of the barrel 560 such that the plunger 570 can reciprocate within the barrel 560 in a longitudinal direction but is restrained by the mating shapes from rotating within the barrel 560. At its proximal end 571, the plunger 570 has a slot 575 for receiving a nut 577 in a manner such that the nut 577 is restrained from rotating relative to the plunger 570, as described above with respect to slot 375 and nut 377. The nut 577 has an internally-threaded surface 578. In an alternative embodiment, an internal surface of the plunger 570 may be internally threaded such that a separate nut 577 is not needed.

The syringe assembly 530 further includes a narrow tube 580, such as a needle or pipette tip. The narrow tube 580 has a proximal end 581 and a distal tip 582. The proximal end 581 of the narrow tube 580 is connected by a hub 584 to the distal end 562 of the barrel 560. The narrow tube 580 has an interior, and the proximal end of the narrow tube interior is in fluid communication with a distal end of the interior 563 of the barrel 560. The plunger 570 and the barrel 560, and in some embodiments also the narrow tube 580, define a fluid chamber 532 within the interior 563 of the barrel 560 and/or the interior of the narrow tube 580. The distal tip 582 of the narrow tube 580 has an opening 583 such that a fluid sample can be drawn into the opening 583 and into the fluid chamber 532, and such that a fluid sample can be expelled from the fluid chamber 532 out of the opening 583.

The syringe assembly 530 also includes a cap 592. The cap 592 has a plurality of resilient tabs 594 which project downwardly. Each of the tabs 594 has an inwardly-facing projection 595 at its end. When assembled, the projections 595 engage the groove 568 of the barrel 560, allowing the cap 592 to be rotated relative to the barrel 560 while restraining separation of the cap 592 from the barrel 560. A screw 597 is attached to the cap 592, extending through a hole in the top of the cap 592. The screw 597 has an externally-threaded surface 598. The head of the screw 597 is on the top side of the cap 592, while a nut 596 on the screw 597 is on the underside of the cap 592, securing the screw 597 to the cap 592. In an alternative embodiment, the cap 592 has an externally-threaded post such that a separate screw 597 is not needed.

The cap 592, screw 597, and nut 577 together form a metering actuator 590 that is configured to control movement of the plunger 570 relative to the barrel 560 in both proximal and distal directions, similar to the metering actuator 390 described above. The externally-threaded surface 598 of the screw 597 is in mating engagement with the internally-threaded surface 578 of the nut 577. When the cap 592 is rotated causing the screw 597 to rotate, the threaded engagement with the nut 577 causes the nut 577 to move longitudinally with respect to the screw 597. This causes the plunger 570 to move longitudinally within the barrel 560.

In use, the operator inserts the collection container assembly 110 into the opening at the distal end 544 of the housing 540 until the collection container stop 117 of the collection container assembly 110 abuts the collection container stop 548 of the housing 540. With the collection container assembly 110 in place, the narrow tube 580 is in contact with the fluid sample, and the operator then rotates the cap 592 in a first direction, drawing the fluid sample from the collection container 111 through the opening 583 of the narrow tube 580 into the fluid chamber 532. The cap 592 is rotated until the proximal end 571 of the plunger 570 abuts a stop within the cap 592, inhibiting further rotation of the cap 592. This full rotation of the cap 592 to the stop position results in moving the plunger 570 proximally relative to the barrel 560 by a predetermined amount. This results in a precise amount of the fluid sample being drawn into the fluid chamber 532. The relative movement between the plunger 570 and the barrel 560 expands the fluid chamber 532, thereby causing the fluid sample to be drawn through the opening 583 of narrow tube 580 into the fluid chamber 532. Once the fluid sample is drawn into the fluid chamber 532, the operator can remove the collection container assembly 110 from the housing 540. At this stage—after the fluid sample has been drawn into the fluid chamber 532 and before the fluid sample is expelled from the fluid chamber 532 into the reaction container—the fluid sample is held in the fluid chamber 532, while the distal tip 582 of the narrow tube 580 remains covered inside the housing 540 proximal to the distal end 544 of the housing 540.

The reaction container assembly 120 is received at the distal end 544 of the housing 540 by the operator inserting the reaction container assembly 120 into the distal end 544 of the housing 540. The projections 127 of the reaction container holder 126 fit into the tracks 545. The operator advances the reaction container assembly 120 into the housing 540 until the projections 127 reach the proximal reaction container stops 546. In this position, the narrow tube 580 extends into the reaction container 121, and the operator then rotates the cap 592 in a second direction, opposite to the first direction. This action expels the fluid sample from the fluid chamber 532 through the opening 583 of the narrow tube 580 into the reaction container 121. The cap 592 is rotated fully until it is inhibited from further rotation. This moves the plunger 570 fully distally within the barrel 560. The relative movement between the plunger 570 and the barrel 560 in this direction contracts the fluid chamber 532, thereby causing the fluid sample to be expelled from the fluid chamber 532 through the opening 583 of the narrow tube 580 into the reaction container 121.

After the fluid sample is expelled into the reaction container 121, the operator moves the reaction container assembly 120 from its proximal position at proximal reaction container stop 546 to its distal position at distal reaction container stop 547. The projections 127 of the reaction container holder 126 are maneuvered (translated and rotated) in the tracks 545 until reaching the distal reaction container stop 547. In this position, the operator can remove the reaction container cover 128 from the reaction container assembly 120. In this condition, the cartridge or syringe assembly 530 holds the reaction container 121 with the fluid sample in it. By handling the syringe assembly 530, the operator can transfer the reaction container 521 to a testing apparatus, such as a polymerase chain reaction machine, to expose the reaction container 121 to one or more processing steps, such as polymerase chain reaction processing steps, while the reaction container 121 remains secured to the syringe assembly 530.

In the embodiment of FIGS. 12A and 12B, the distal tip of the narrow tube 580 is configured to remain at all times located inside the housing 540 proximal to the distal end 544 of the housing 540. Also, during part of the operation, the distal tip of the narrow tube 580 is located inside the collection container 111 or located inside the reaction container 121.

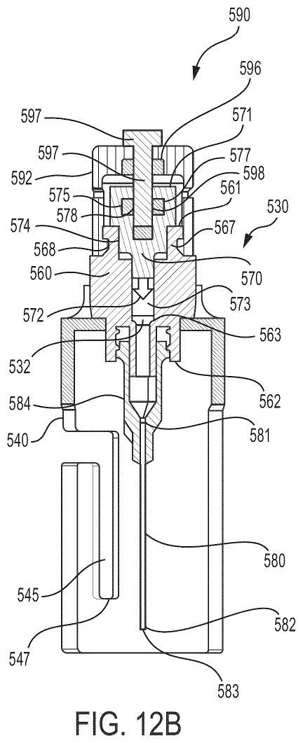

FIGS. 13 through 21 illustrate a fourth embodiment of a set 700 of components including a cartridge or syringe assembly 730 for transferring a fluid sample for testing. The set 700 of components includes the syringe assembly 730, a collection container assembly 710, and a reaction container assembly 720.

As shown in FIG. 13, the collection container assembly 710 includes collection container 711 in which a fluid sample, such as a sample of saliva or blood from a patient, may be collected. The collection container has an opening 712 at its top end. The opening 712 is covered by a pierceable cover 714 that is pierceable by a narrow tube such as a needle or pipette tip to access the contents of the collection container 711. The collection container assembly 710 may also include a cap 716 that fits over the top of the collection container 711, for example by a threaded fit, friction fit, snap fit, or other engagement.

The reaction container assembly 720 comprises a reaction container 721 having an opening 722 at its top end. The opening 722 is covered by a pierceable cover 723 that is pierceable by a narrow tube such as a needle or pipette tip to dispense a sample into the reaction container 721. The reaction container assembly 720 also comprises a reaction container holder 724 for holding the reaction container 721. The reaction container holder 724 has one or more resilient tabs 725 extending upwardly away from the reaction container 721. Each of the resilient tabs 725 has an outwardly-extending projection 726 at its end. The reaction container assembly 720 further includes a protective reaction container cap or cover 728.

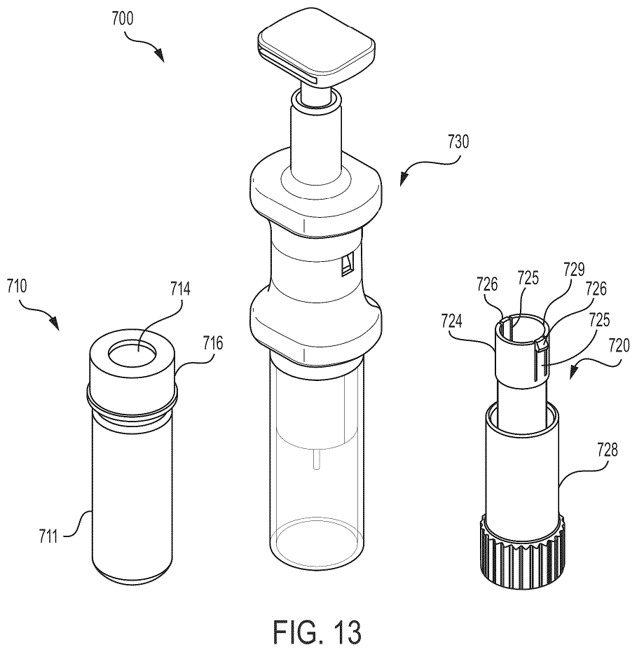

FIGS. 14A and 14B show side and cross-sectional views, respectively, of the syringe assembly 730. The syringe assembly 730 includes a housing 740 having a side wall 742, a proximal end 743, and a distal end 744. In this embodiment, the housing 740 includes a removable housing cover 750 for covering a narrow tube 780, such as a needle or pipette tip. The housing also includes a middle part 751 and an upper part 753. The middle part 751 has at least a portion with a diameter sized to fit with the housing cover 750, such as with a sliding friction fit, so that the middle part 751 and housing cover 750 may be secured together while allowing the housing cover 750 to be removed from middle part 751. The distal end 752 of the middle part 751 of the housing 740 also serves as a collection container stop 748, and the distal end 752 may have a beveled surface, which can facilitate centering of and engagement with the collection container assembly 710. The housing 740 includes a lower ledge 754 between the upper part 753 and the middle part 751 with an opening for accommodating the barrel 760. The housing 740 also includes an upper ledge 755 for positioning a spring 768.

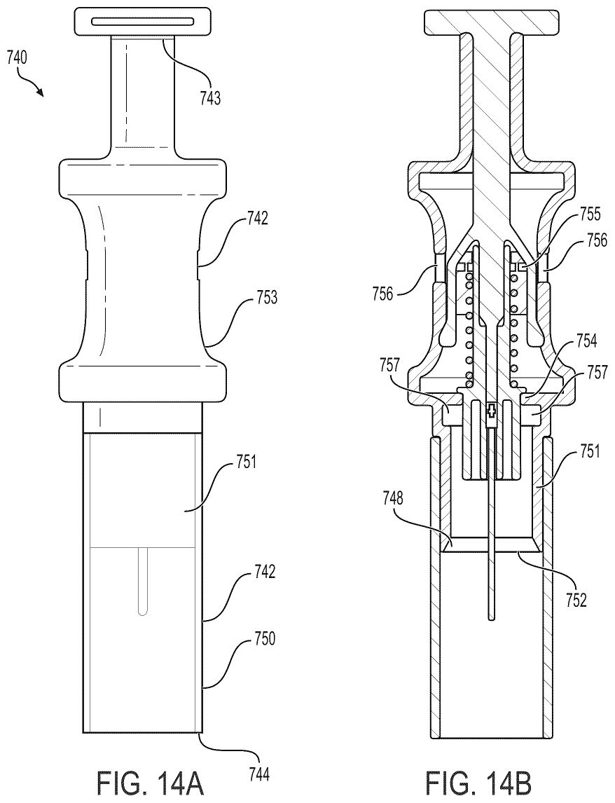

As shown in FIG. 15B, the syringe assembly 730 also includes a syringe barrel 760. The syringe barrel 760 has a proximal end 761, a distal end 762, and an interior 763. The barrel 760 also has an outwardly-projecting tab or flange 766. When the syringe assembly 730 is assembled, the flange 766 is biased by the spring 768 against the proximal side of the lower ledge 754. The lower end of the spring 768 presses against the flange 766 of the barrel 760, while the upper end of the spring 768 presses against the upper ledge 755 of the housing 740. When the syringe assembly 730 is assembled, at least the distal end 762 of the barrel 760 is located inside the housing 740.

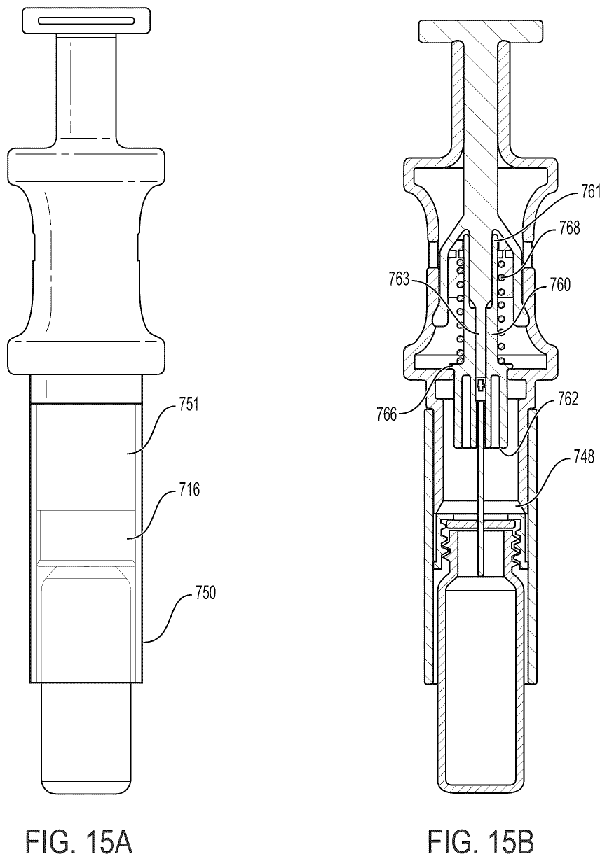

The syringe assembly 730 also includes a plunger 770. As shown in FIG. 16B, the plunger 770 has a proximal end 771 and a distal end 772. The plunger 770 has a stopper 773 at its distal end 772. The plunger 770 is reciprocally moveable in the interior 763 of the barrel 760 in a proximal direction and in a distal direction. The plunger 770 further includes one or more resilient arms 774, with each resilient arm 774 having an outwardly-facing projection 775 at its end.

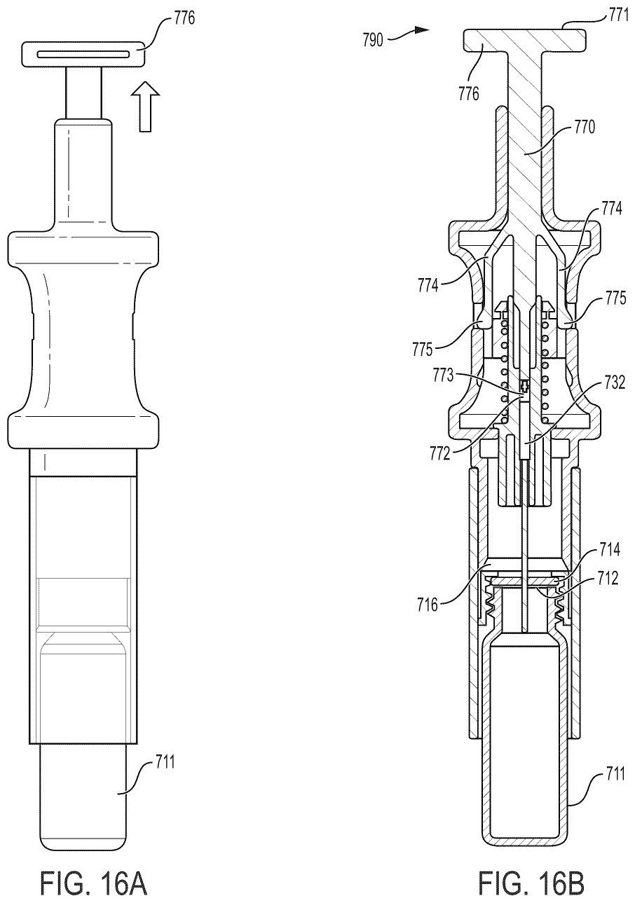

The syringe assembly 730 further includes a narrow tube 780, such as a needle or pipette tip. As shown in FIG. 17B, the narrow tube 780 has a proximal end 781 and a distal tip 782. The proximal end 781 of the narrow tube 780 is connected to the distal end 762 of the barrel 760. The narrow tube 780 has an interior, and the proximal end of the narrow tube interior is in fluid communication with a distal end of the interior 763 of the barrel 760. The plunger 770 and the barrel 760, and in some embodiments also the narrow tube 780, define a fluid chamber 732 within the interior 763 of the barrel 760 and/or the interior of the narrow tube 780. The distal tip 782 of the narrow tube 780 has an opening 783 such that a fluid sample can be drawn through the opening 783 and into the fluid chamber 732, and such that a fluid sample can be expelled from the fluid chamber 732 out of the opening 783.

The upper part 753 of the housing 740 has one or more recesses or openings 756 for receiving the projection(s) 775 of the plunger 770 when the plunger 770 is moved to a proximal position. The proximal end 771 of the plunger 770 has a knob 776 for gripping the plunger 770. The knob 776, projection(s) 775 and recess(es) or opening(s) 756 together form a metering actuator 790 that is configured to control movement of the plunger 770 relative to the barrel 760 at least in a proximal direction.

The barrel 760, the plunger 770, and the narrow tube 780 are configured such that relative movement between the plunger 770 and the barrel 760 that expands the fluid chamber 732 causes a fluid sample to be drawn through the opening 783 of the narrow tube 780 into the fluid chamber 732. Similarly, the barrel 760, the plunger 770, and the narrow tube 780 are configured such that relative movement between the plunger 770 and the barrel 760 that contracts the fluid chamber 732 causes the fluid sample to be expelled from the fluid chamber 732 through the opening 783 of the narrow tube 780.

FIGS. 14A through 21 show various stages in transferring a fluid sample for testing. FIGS. 14A and 14B show the syringe assembly 730 in a neutral position, prior to receiving the sample. The plunger 770 is in the distal position, with the knob 776 in its distal-most position. The resilient arms 774 are biased inwardly by the inner surface of the upper part 753 of the housing 740.

FIGS. 15A through 16B show stages in drawing a fluid sample from the collection container 711. A fluid sample has been collected from a patient and is in the collection container 711, optionally along with a buffer if desired for the particular application. The operator inserts the collection container assembly 710 into the opening at the distal end 744 of the housing 740 until the top of the collection container assembly 710 abuts the collection container stop 748 at the distal end 752 of the middle part 751 of the housing 140, as shown in FIGS. 15A and 15B. This limits further motion of the collection container assembly 710 into the housing 740. The cap 716 may slide within the housing cover 750 of the housing 740 with a slight friction fit to aid in keeping the collection container assembly 710 in place.

With the collection container assembly 710 in place, the narrow tube 780 is in the collection container 711 in contact with the fluid sample. The operator then moves the knob 776 proximally from the first or distal position as shown in FIGS. 14A through 15B to the second or proximal position as shown in FIGS. 16A and 16B. The proximal movement of the knob 776 pulls the plunger 770 proximally. When the projections 775 reach the recesses or openings 756 in the housing side wall 742, the spring force of the resilient arms 774 presses the projections 775 into the recesses or openings 756. This locks the plunger 770 relative to the housing 740 and inhibits further proximal movement of the plunger 770 relative to the barrel 760. Moving the knob 776 from the first position to the second position, in which the projections 775 engage the recesses or openings 756, results in moving the plunger 770 proximally relative to the barrel 760 by a predetermined amount, defined by the longitudinal distance between the first position and the second position. This results in a precise amount of the fluid sample being drawn into the fluid chamber 732. The relative movement between the plunger 770 and the barrel 760 expands the fluid chamber 732, thereby causing the fluid sample to be drawn through the opening 783 of the narrow tube 780 into the fluid chamber 732. Once the fluid sample is drawn into the fluid chamber 732, the operator can remove the collection container assembly 710, as shown in FIGS. 17A and 17B. At this stage—after the fluid sample has been drawn into the fluid chamber 732 and before the fluid sample is expelled from the fluid chamber 732 into the reaction container—the fluid sample is held in the fluid chamber 732, while the distal tip 782 of the narrow tube 780 remains covered inside the housing 740 proximal to the distal end 744 of the housing 740, as can be seen in FIGS. 17A and 17B.

As noted above, in some embodiments, the plunger moves proximally relative to the barrel (and/or a narrow tube within the barrel) as is defined by a longitudinal distance, which can precisely control the amount (e.g., volume) of fluid drawn into a fluid chamber of the barrel (and/or a narrow tube within the barrel). In some embodiments, the longitudinal distance is greater than or equal to 1 μm, greater than or equal to 10 μm, greater than or equal to 20 μm, greater than or equal to 50 μm, greater than or equal to 75 μm, greater than or equal to 100 μm, greater than or equal to 200 μm, greater than or equal to 250 μm, greater than or equal to 300 μm, greater than or equal to 500 μm, greater than or equal to 700 μm, greater than or equal to 750 μm, greater than or equal to 1 mm, or greater than or equal to 2 mm. In some embodiments, the longitudinal distance is less than or equal to 2 mm, less than or equal to 1 mm, less than or equal to 750 μm, less than or equal to 700 μm, less than or equal to 500 μm, less than or equal to 300 μm, less than or equal to 250 μm, less than or equal to 200 μm, less than or equal to 100 μm, less than or equal to 50 μm, less than or equal to 20 μm, less than or equal to 10 μm, or less than or equal to 1 μm. Combinations of the above-referenced ranges are also possible (e.g., greater than or equal to 1 μm and less than or equal to 2 mm). Other ranges are possible.

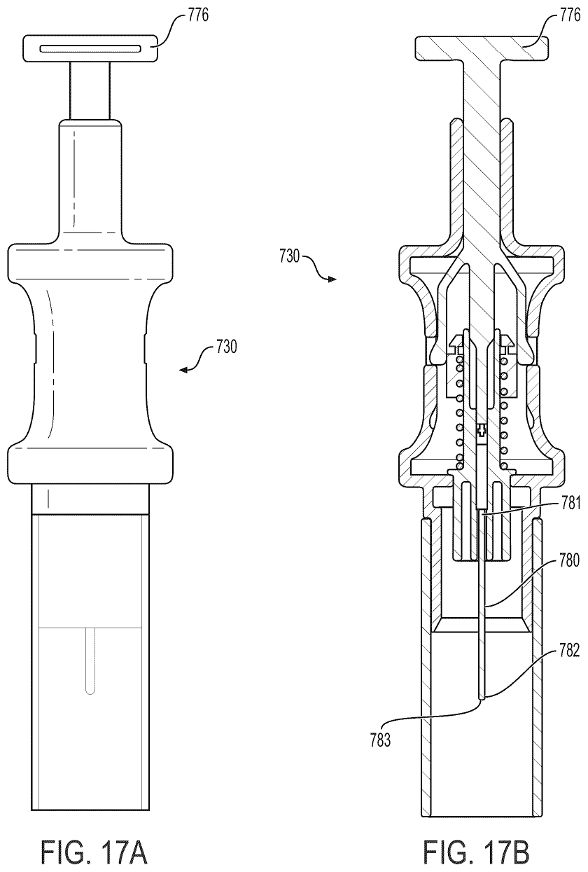

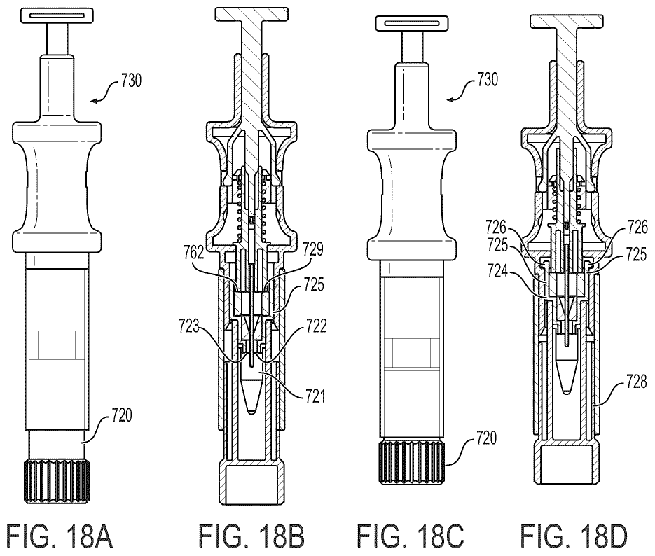

FIGS. 18A through 18D show stages in expelling the fluid sample from the fluid chamber 732 into the reaction container 721. The reaction container 721 may have reagents in it for a desired application. The reaction container assembly 720 is received at the distal end 744 of the housing 740 by the operator inserting the reaction container assembly 720 into the distal end 744 of the housing 740. FIG. 18A shows a side view and FIG. 18B shows a cross-section view at a stage at which the reaction container assembly 720 has been advanced until a proximal surface 729 of the reaction container holder 724 abuts against the distal end 762 of the barrel 760. The resilient tabs 725 of the reaction container holder 724 are biased inwardly by the inner surface of the middle part 751 of the housing 740. As can be seen in FIG. 18B, advancement of the reaction container assembly 720 to this position causes the narrow tube 780 to extend into the reaction container 721.

From the position shown in FIGS. 18A and 18B, as the operator continues to move the reaction container assembly 720 proximally into the housing 740, further advancement of the reaction container assembly 720 presses the barrel 760 proximally. The proximal surface 729 of the reaction container holder 724 pushes against the distal end 762 of the barrel 760, or against one or more intermediate parts that push(es) against the barrel 760 (e.g., a pipette), causing the barrel 760 to move proximally relative to the housing 740. As this happens, the spring 768 becomes further compressed as the tab or flange 766 of the barrel limiting one end of the spring 768 moves toward the ledge 755 of the housing 740 limiting the other end of the spring 768. The operator continues to move the reaction container assembly 720 proximally into the housing 740 until the projections 726 on the resilient tabs 725 of the reaction container holder 724 reach the recess(es) or opening(s) 757 in the middle part 751 of the housing 740. When the projections 726 reach the recess(es) or opening(s) 757 in the housing side wall 742, the spring force of the resilient tabs 725 presses the projections 726 into the recess(es) or opening(s) 757, as shown in FIG. 18D. This locks the reaction container holder 724 relative to the housing 740 and inhibits further movement of the reaction container holder 724 relative to the housing 740.

Moving the reaction container assembly 720 from the position shown in FIGS. 18A and 18B to the position shown in FIGS. 18C and 18D results in moving the barrel 760 proximally relative to the plunger 770. The relative movement between the plunger 770 and the barrel 760 in this direction contracts the fluid chamber 732, thereby causing the fluid sample to be expelled from the fluid chamber 732 through the opening 783 of the narrow tube 780 into the reaction container 721.

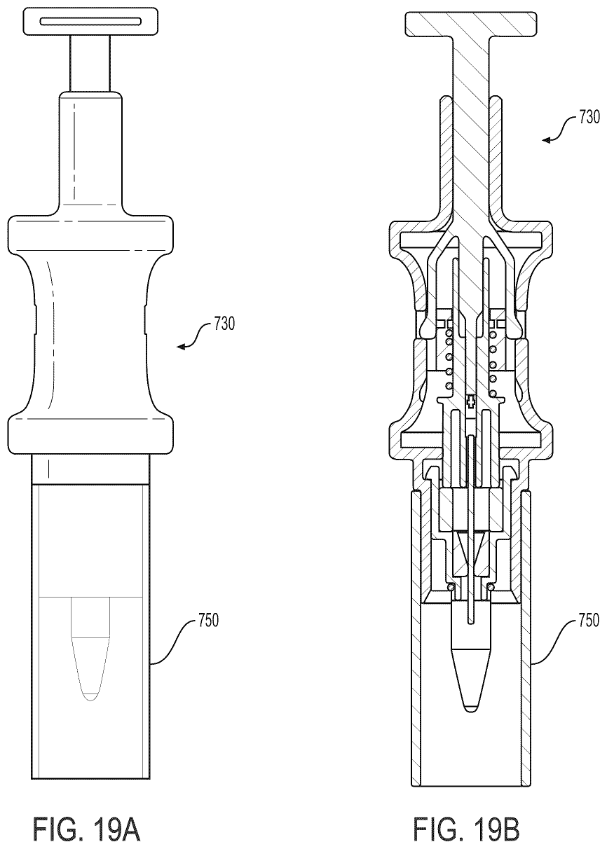

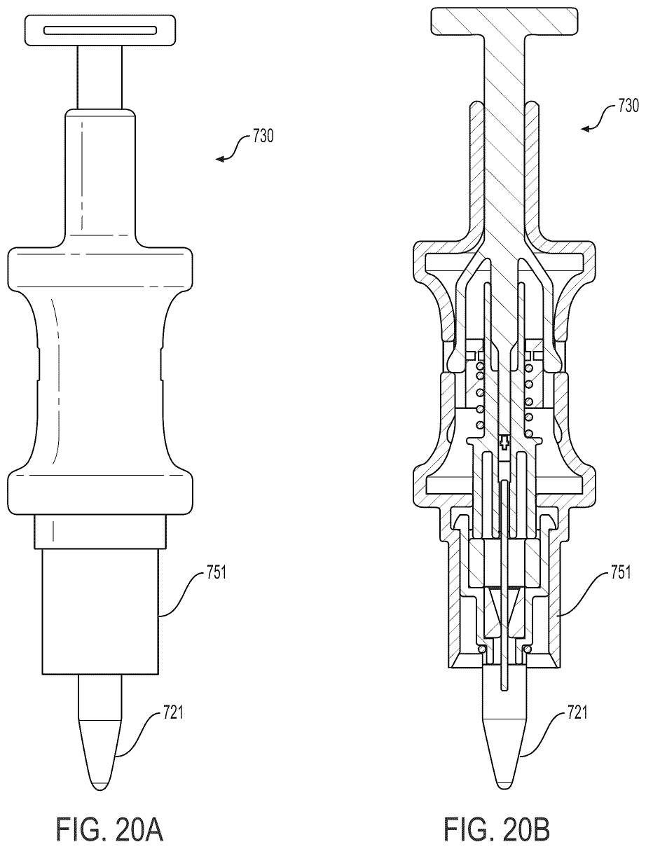

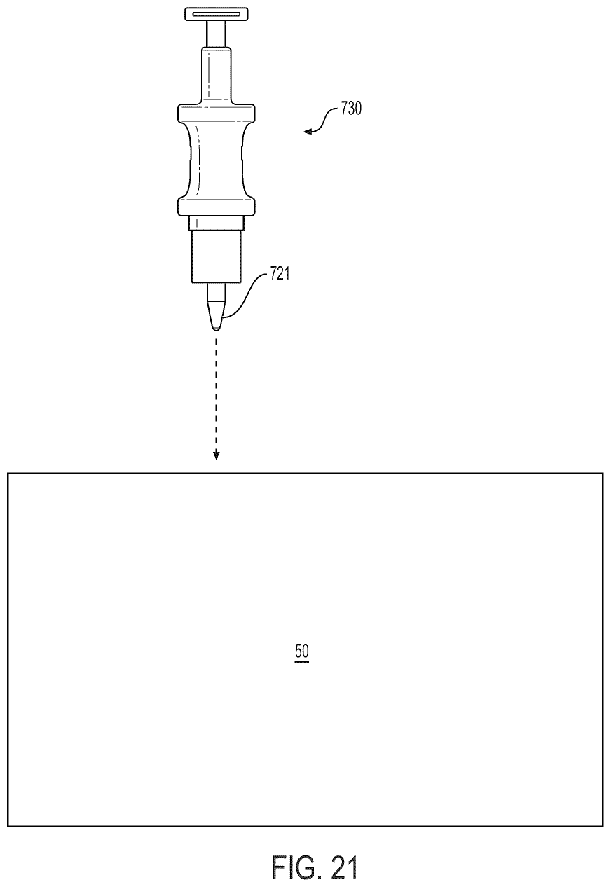

FIGS. 19A through 21 show further stages in use of the syringe assembly 730. After the fluid sample is expelled into the reaction container 721, the operator removes the reaction container cover 728, as shown in FIGS. 19A and 19B, as well as the housing cover 750 of the housing 740, as shown in FIGS. 20A and 20B. In this condition, the cartridge or syringe assembly 730 holds the reaction container 721 with the fluid sample in it. By handling the syringe assembly 730, the operator can transfer the reaction container 721 to a testing apparatus 50, as shown in FIG. 21, such as a polymerase chain reaction machine, to expose the reaction container 721 to one or more processing steps, such as polymerase chain reaction processing steps, while the reaction container 721 remains secured to the syringe assembly 730.

In the embodiment of FIGS. 13 through 21, the distal tip of the narrow tube 780 is configured to remain at all times located inside the housing 740 proximal to the distal end 744 of the housing 740 or located inside the reaction container 721. For example, in FIGS. 20A and 20B, after the housing cover 750 of the housing 740 is removed, the distal tip of the narrow tube 780 is located beyond the distal end of the housing 740 but is located inside the reaction container 721 and is thereby protected from exposure to a person handling the device.

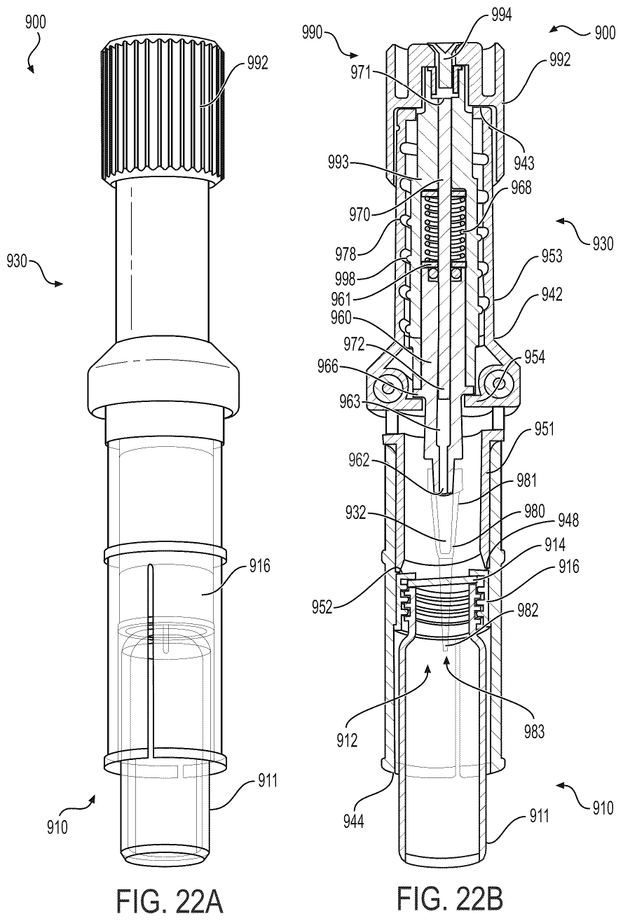

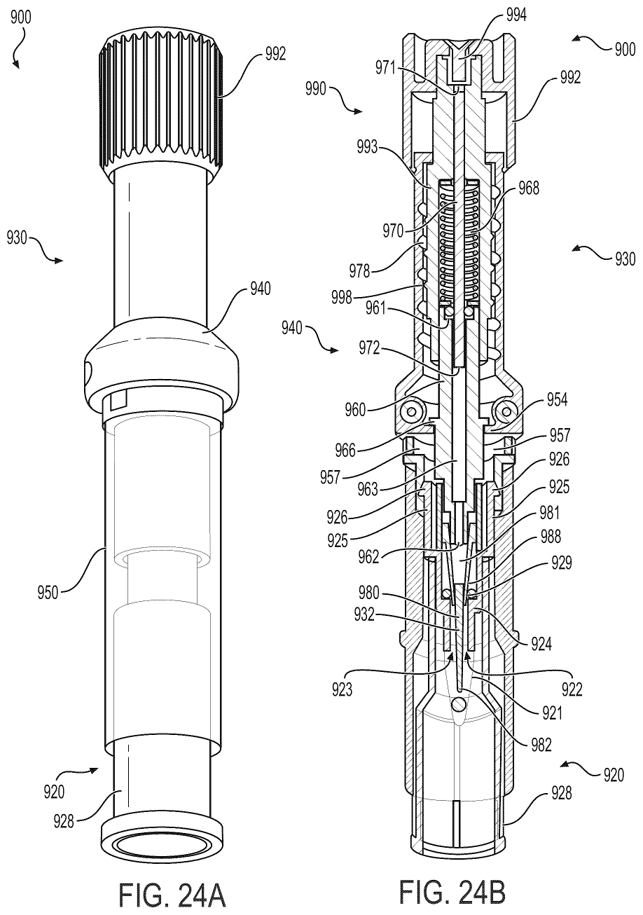

FIGS. 22A through 26 illustrate a fifth embodiment of a set 900 of components including a cartridge or syringe assembly 930 for transferring a fluid sample for testing. In this embodiment, the set 900 of components includes the syringe assembly 930, a collection container assembly 910, and a reaction container assembly 920 (shown in FIGS. 24A and 24B).

As shown in FIG. 22B, the collection container assembly 910 includes collection container 911 in which a fluid sample, such as a sample of saliva or blood from a patient, may be collected. The collection container has an opening 912 at its top end. The opening 912 is covered by a pierceable cover 914 that is pierceable by a narrow tube such as a needle or pipette tip to access the contents of the collection container 911. The collection container assembly 910 may also include a cap 916 that fits over the top of the collection container 911, for example by a threaded fit, friction fit, snap fit, or other engagement.

The reaction container assembly 920 comprises a reaction container 921 having an opening 922 at its top end. The opening 922 is covered by a pierceable cover 923 that is pierceable by a narrow tube such as a needle or pipette tip to dispense a sample into the reaction container 921. The reaction container assembly 920 also comprises a reaction container holder 924 for holding the reaction container 921. The reaction container holder 924 has one or more resilient tabs 925 extending upwardly away from the reaction container 921. Each of the resilient tabs 925 has an outwardly-extending projection 926 at its end. The reaction container assembly 920 further includes a protective reaction container cap or cover 928.

The syringe assembly 930 includes a housing 940 having a side wall 942, a proximal end 943, and a distal end 944. In this embodiment, the housing 940 includes a removable housing cover 950 for covering a narrow tube 980, such as a needle or pipette tip. The housing 940 also includes a middle part 951 and an upper part 953. The middle part 951 has at least a portion with a diameter sized to fit with the housing cover 950, such as with a sliding friction fit, so that the middle part 951 and housing cover 950 may be secured together while allowing the housing cover 950 to be removed from middle part 951. The distal end 952 of the middle part 951 of the housing 940 may also serve as a collection container stop 948, and the distal end 952 may have a beveled surface, which can facilitate centering of and engagement with the collection container assembly 910. The housing 940 includes a lower ledge 954 between the upper part 953 and the middle part 951 with an opening for accommodating the syringe barrel 960. A spring 968 may bias the syringe barrel 960 distally.

The syringe barrel 960 has a proximal end 961, a distal end 962, and an interior 963. The barrel 960 also has an outwardly-projecting tab or flange 966. When the syringe assembly 930 is assembled, the flange 966 is biased by the spring 968 against the proximal side of the lower ledge 954. The lower end of the spring 968 presses against the barrel 960, while the upper end of the spring 968 presses against a sleeve 993. When the syringe assembly 930 is assembled, at least the distal end 962 of the barrel 960 is located inside the housing 940.

The syringe assembly 930 also includes a plunger 970. The plunger 970 has a proximal end 971 and a distal end 972. The plunger 970 may have a stopper at its distal end 972. The plunger 970 is reciprocally moveable in the interior 963 of the barrel 960 in a proximal direction and in a distal direction.

The syringe assembly 930 further includes a narrow tube 980, such as a needle or pipette tip. The narrow tube 980 has a proximal end 981 and a distal tip 982. The proximal end 981 of the narrow tube 980 is connected to the distal end 962 of the barrel 960. The narrow tube 980 has an interior, and the proximal end of the narrow tube interior is in fluid communication with a distal end of the interior 963 of the barrel 960. The plunger 970 and the barrel 960, and in some embodiments also the narrow tube 980, define a fluid chamber 932 within the interior 963 of the barrel 960 and/or the interior of the narrow tube 980. The distal tip 982 of the narrow tube 980 has an opening 983 such that a fluid sample can be drawn through the opening 983 and into the fluid chamber 932, and such that a fluid sample can be expelled from the fluid chamber 932 out of the opening 983.

The syringe assembly 930 also includes a cap 992. The cap 992 is connected to or is integral with the sleeve 993 such that the sleeve 993 rotates with the cap 992. In the illustrated embodiment, the cap 992 is connected to the sleeve 993 by a fastener 994, but other variations are possible, such as other types of connections or integrally molding the parts. The sleeve 993 extends inside the housing 940 and has an externally-threaded surface 998 that threadingly engages an internally-threaded surface 978 that is a part of or is fixed relative to the housing 940. In an alternative embodiment, the cap 992 is connected to or is integral with a sleeve that extends around the outside of the housing 940, wherein the sleeve has an internally-threaded surface that threadingly engages an externally-threaded surface that is a part of or is fixed relative to the housing 940. The cap 992 is rotatable relative to the housing 940, and the sleeve 993 rotates with the cap 992. Due to the threaded engagement between the sleeve 993 and the housing 940, when the cap 992 is rotated, the cap 992 and sleeve 993 move longitudinally with respect to the housing 940.

The cap 992 and sleeve 993 are arranged such that longitudinal movement of the cap 992 and sleeve 993 relative to the housing 940, which occurs by rotation of the cap 992, also causes longitudinal movement of the plunger 970. The plunger 970 may be connected to the cap 992, to the sleeve 993, or to another part connected to the cap 992 or sleeve 993. The connection may be rigid such that the plunger 970 rotates with the cap 992 and sleeve 993. In some embodiments, the sleeve 993 and plunger 970 may be a single integral piece. Alternatively, the cap 992 and sleeve 993 may carry the plunger 970 for longitudinal movement with the cap 992 and sleeve 993 but without rotational movement of the plunger 970. For example, the sleeve 993 may have a flange that engages a flange of the plunger 970, such that when the sleeve 993 moves longitudinally it causes longitudinal movement of the plunger 970 but rotation of the sleeve 993 does not cause rotation of the plunger 970. An o-ring (shown in FIG. 22B) around the plunger 970 at the proximal end of the barrel 960 can facilitate sealing.

The cap 992, sleeve 993, and the threaded surfaces 978, 998 together form a metering actuator 990 that is configured to control movement of the plunger 970 relative to the barrel 960. Rotation of the cap 992 causes rotation of the sleeve 993, because the sleeve 993 is secured to or integral with the cap 992. The threaded surface 998 of the sleeve 993 is in mating engagement with the threaded surface 978 associated with the housing 940. Thus, when the cap 992 is rotated causing the sleeve 993 to rotate, the threaded engagement with the housing 940 causes the cap 992 and sleeve 993 to move longitudinally with respect to the housing 940. This causes the plunger 970 to move longitudinally within the barrel 960.

The barrel 960, the plunger 970, and the narrow tube 980 are configured such that relative movement between the plunger 970 and the barrel 960 that expands the fluid chamber 932 causes a fluid sample to be drawn through the opening 983 of the narrow tube 980 into the fluid chamber 932. Similarly, the barrel 960, the plunger 970, and the narrow tube 980 are configured such that relative movement between the plunger 970 and the barrel 960 that contracts the fluid chamber 932 causes the fluid sample to be expelled from the fluid chamber 932 through the opening 983 of the narrow tube 980.

FIGS. 22A through 26 show various stages in transferring a fluid sample for testing. FIGS. 22A and 22B show the syringe assembly 930 in a neutral position, prior to receiving the sample. The plunger 970 is in the distal position, with the cap 992 in its distal-most position. A fluid sample has been collected from a patient and is in the collection container 911, optionally along with a buffer if desired for the particular application. The operator inserts the collection container assembly 910 into the opening at the distal end 944 of the housing 940 until the top of the collection container assembly 910 abuts the collection container stop 948 at the distal end 952 of the middle part 951 of the housing 940, as shown in FIG. 22B. This limits further motion of the collection container assembly 910 into the housing 940. The cap 916 and/or collection container 911 may slide within the housing cover 950 of the housing 940 with a slight friction fit to aid in keeping the collection container assembly 910 in place.

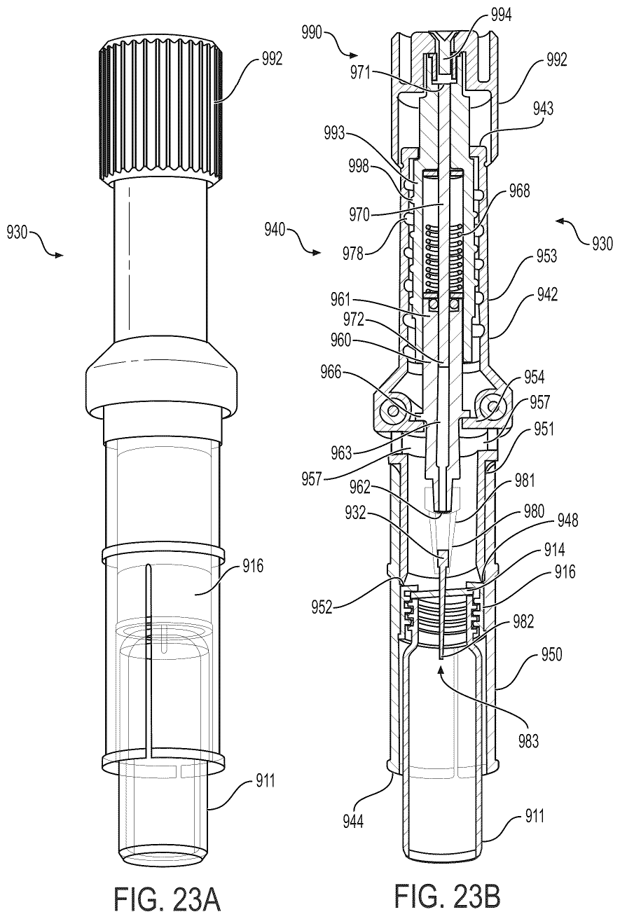

With the collection container assembly 910 in place, as shown in FIGS. 22A and 22B, the narrow tube 980 is in the collection container 911 in contact with the fluid sample. The operator then rotates the cap 992 in a first direction, thereby rotating the sleeve 993 and, due to the engagement of threaded surfaces 998 and 978, causing the cap 992 and sleeve 993 to move longitudinally in a proximal direction to the position shown in FIGS. 23A and 23B. The proximal movement of the cap 992 and sleeve 993 pulls the plunger 970 proximally. When the cap 992 and sleeve 993 reach the limit of their travel, which may be provided by a suitable stop, further proximal movement of the plunger 970 relative to the barrel 960 is stopped. Moving the cap 992 and sleeve 993 from the first position shown in FIGS. 22A and 22B to the second position shown in FIGS. 23A and 23B results in moving the plunger 970 proximally relative to the barrel 960 by a predetermined amount, defined by the longitudinal distance between the first position and the second position. This results in a precise amount of the fluid sample being drawn from the collection container 911 into the fluid chamber 932. The relative movement between the plunger 970 and the barrel 960 expands the fluid chamber 932, thereby causing the fluid sample to be drawn through the opening 983 of the narrow tube 980 into the fluid chamber 932.

Once the fluid sample is drawn into the fluid chamber 932, the operator can remove the collection container assembly 910. At this stage—after the fluid sample has been drawn into the fluid chamber 932 and before the fluid sample is expelled from the fluid chamber 932 into the reaction container—the fluid sample is held in the fluid chamber 932, while the distal tip 982 of the narrow tube 980 remains covered inside the housing 940 proximal to the distal end 944 of the housing 940.

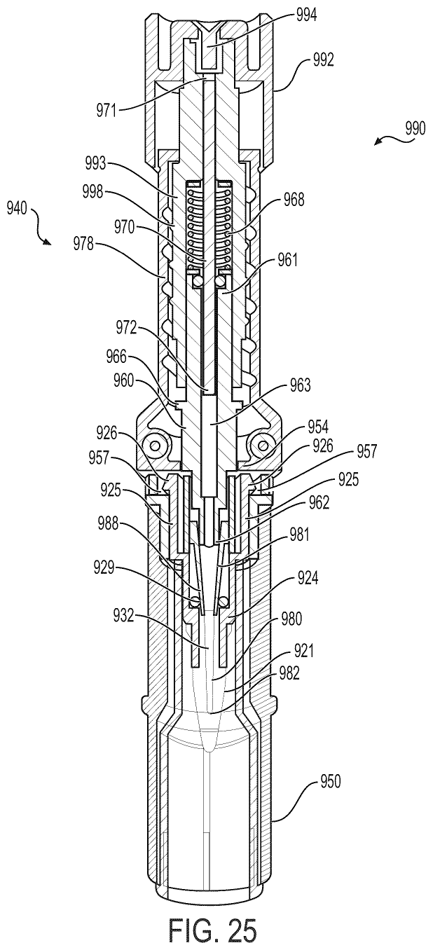

FIGS. 24A through 25 show stages in expelling the fluid sample from the fluid chamber 932 into the reaction container 921. The reaction container 921 may have reagents in it for a desired application. The reaction container assembly 920 is received at the distal end 944 of the housing 940 by the operator inserting the reaction container assembly 920 into the distal end 944 of the housing 940. FIG. 24A shows a side view and FIG. 24B shows a cross-section view at a stage at which the reaction container assembly 920 has been advanced until a surface 929 of the reaction container holder 924, which may be a surface of a seal, o-ring, ledge, or other surface, abuts against a surface 988 of the narrow tube 980 or of the barrel 960 (or a surface of another part that is connected to the barrel 960). The resilient tabs 925 of the reaction container holder 924 are biased inwardly by the inner surface of the middle part 951 of the housing 940. As can be seen in FIG. 24B, advancement of the reaction container assembly 920 to this position causes the narrow tube 980 to extend into the reaction container 921.

From the position shown in FIGS. 24A and 24B, as the operator continues to move the reaction container assembly 920 proximally into the housing 940, further advancement of the reaction container assembly 920 presses the barrel 960 proximally. The surface 929 of the reaction container holder 924 pushes against the surface 988 of the narrow tube 980, causing the barrel 960 to move proximally relative to the housing 940. As this happens, the spring 968 becomes further compressed as the surface of the barrel 960 limiting one end of the spring 968 moves toward the surface of the sleeve 993 limiting the other end of the spring 968. The operator continues to move the reaction container assembly 920 proximally into the housing 940 until the projections 926 on the resilient tabs 925 of the reaction container holder 924 reach the recess(es) or opening(s) 957 in the middle part 951 of the housing 940. When the projections 926 reach the recess(es) or opening(s) 957 in the housing side wall 942, the spring force of the resilient tabs 925 presses the projections 926 into the recess(es) or opening(s) 957, as shown in FIG. 25. This locks the reaction container holder 924 relative to the housing 940 and inhibits further movement of the reaction container holder 924 relative to the housing 940.

Moving the reaction container assembly 920 from the position shown in FIGS. 24A and 24B to the position shown in FIG. 25 results in moving the barrel 960 proximally relative to the plunger 970. The relative movement between the plunger 970 and the barrel 960 in this direction contracts the fluid chamber 932, thereby causing the fluid sample to be expelled from the fluid chamber 932 through the opening 983 of the narrow tube 980 into the reaction container 921.

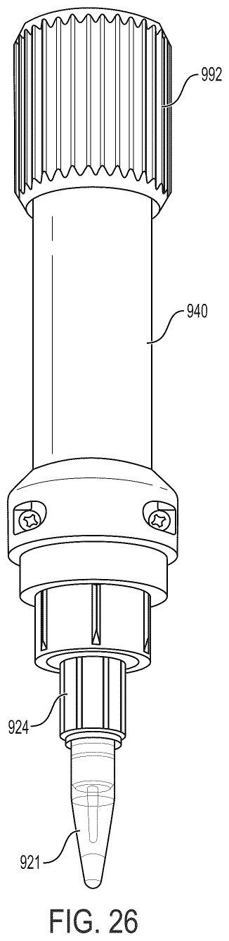

After the fluid sample is expelled into the reaction container 921, the operator removes the reaction container cover 928 as well as the housing cover 950 of the housing 940, as shown in FIG. 26. In this condition, the cartridge or syringe assembly 930 holds the reaction container 921 with the fluid sample in it. The reaction container 921 remains locked to the housing 940 by the engagement of the projections 926 of the reaction container holder 924 at the recess(es) or opening(s) 957. By handling the syringe assembly 930, the operator can transfer the reaction container 921 to a testing apparatus (e.g., testing apparatus 50 in FIG. 21), such as a polymerase chain reaction machine, to expose the reaction container 921 to one or more processing steps, such as polymerase chain reaction processing steps, while the reaction container 921 remains secured to the syringe assembly 930.

In the embodiment of FIGS. 22A through 26, the distal tip of the narrow tube 980 is configured to remain at all times located inside the housing 940 proximal to the distal end 944 of the housing 940 or located inside the reaction container 921. For example, in FIG. 26, after the housing cover 950 of the housing 940 is removed, the distal tip of the narrow tube 980 is located beyond the distal end of the housing 940 but is located inside the reaction container 921 and is thereby protected from exposure to a person handling the device.

To facilitate visualization by the user, various components may be transparent. For example, the housing covers and/or container covers may be transparent. For example, the reaction container cover 928 and/or the housing cover 950 may be transparent.

Various components described herein may be made as separate or integral components. As one example, in some embodiments, the barrel and narrow tube (e.g., barrel 960 and narrow tube 980) may be separate components or manufactured as a single integral piece.

Embodiments in accordance with the disclosed subject matter may use any suitable materials. Some components, such as collection containers, reaction containers, and syringe barrels and plungers in certain embodiments, may be available as off-the-shelf parts. Components may be made by any suitable manufacturing method. For example, some components may be injection molded from a suitable material, such as polypropylene.

Embodiments in accordance with the disclosed subject matter may be used for transferring samples (e.g., blood, saliva, urine, stool) for various different testing procedures. In some embodiments, the sample is a biological sample. As just one of many examples, some embodiments may be used for the preparation of fluid samples for the amplification and detection of viruses, such as the SARS-COV-2 RNA in clinical saliva samples. The reagents in the reaction container may be lyophilized RT-PCR reagents. When a saliva sample is transferred to the reaction container, the sample reconstitutes the lyophilized reagents. At this point the cartridge with the reaction container attached can be inserted into a suitable instrument for ultrafast RT-PCR for processing steps. However, it will be understood that other samples are possible, such as non-biological samples. For example, some embodiments may be used to collect a sample from the environment, such as wastewater, contaminated water, or other samples from the environment.

While many of the embodiments described herein describe a reaction container containing RT-PCT reagents, it should be understood that other reaction types are contemplated. For example, the reaction container may be configured to (e.g., contain reagent for) a variety of chemical and/or biological reactions. Non-limiting examples of chemical and/or biological reactions include acid-base reactions, oxidation-reduction reactions, precipitation reactions, enzymatic reactions, and/or antigen-antibody binding. Of course, other chemical and/or biological reactions are contemplated as this disclosure is not so limited. Those skilled in the art, in view of the present disclosure, will be capable of determining adequate chemical and/or biological systems to be used with the various embodiments described herein.

As will be appreciated from the disclosure, some embodiments in accordance with the disclosed subject matter enable drawing a precise, fixed microvolume of a fluid sample into the syringe barrel and transferring that precise, fixed microvolume to a reaction container. Example amounts may be 1 microliter or less to 100 milliliters or more; as one example, the transferred microvolume may be 20 microliters +/−2 microliters. In some embodiments, the volume to be held or transferred (e.g., within a narrow tube, within a fluid chamber) may be greater than or equal to 1 microliter, greater than or equal to 2 microliters, greater than or equal to 5 microliters, greater than or equal to 10 microliters, greater than or equal to 12 microliters, greater than or equal to 15 microliters, greater than or equal to 20 microliters, greater than or equal to 25 microliters, greater than or equal to 30 microliters, greater than or equal to 50 microliters, greater than or equal to 75 microliters, greater than or equal to 100 microliters, greater than or equal to 250 microliters, greater than or equal to 300 microliters, greater than or equal to 400 microliters, greater than or equal to 500 microliters, greater than or equal to 600 microliters, greater than or equal to 750 microliters, greater than or equal to 1 milliliter, greater than or equal to 1.3 milliliters, greater than or equal to 1.5 milliliters, greater than or equal to 2 milliliters, greater than or equal to 5 milliliters, greater than or equal to 10 milliliters, greater than or equal to 20 milliliters, greater than or equal to 30 millimeters, greater than or equal to 50 milliliters, greater than or equal to 70 milliliters, greater than or equal to 90 milliliters or greater than or equal to 100 milliliters. In some embodiments, the volume to be held or transferred may be less than or equal to 100 milliliters, less than or equal to 90 milliliters, less than or equal to 70 milliliters, less than or equal to 50 milliliters, less than or equal to 30 millimeters, less than or equal to 20 milliliters, less than or equal to 10 milliliters, less than or equal to 5 milliliters, less than or equal to 10 milliliters, less than or equal to 5 milliliters, less than or equal to 2 milliliters, less than or equal to 1.5 milliliters, less than or equal to 1.3 milliliters, less than or equal to 1 milliliter, less than or equal to 750 microliters, less than or equal to 600 microliters, less than or equal to 500 microliters, less than or equal to 400 microliters, less than or equal to 300 microliters, less than or equal to 250 microliters, less than or equal to 100 microliters, less than or equal to 75 microliters, less than or equal to 50 microliters, less than or equal to 30 microliters, less than or equal to 25 microliters, less than or equal to 20 microliters, less than or equal to 15 microliters, less than or equal to 12 microliters, less than or equal to 10 microliters, less than or equal to 5 microliters, less than or equal to 2 microliters, or less than or equal to 1 microliter. Combinations of the above-referenced ranges are also possible (e.g., greater than or equal to 1 microliter and less than or equal to 100 microliters). Other ranges are possible.

In some embodiments, the internal volume (e.g., holding volume) of the narrow tube may be greater than or equal to 1 microliter, greater than or equal to 2 microliters, greater than or equal to 5 microliters, greater than or equal to 10 microliters, greater than or equal to 12 microliters, greater than or equal to 15 microliters, greater than or equal to 20 microliters, greater than or equal to 25 microliters, greater than or equal to 30 microliters, greater than or equal to 50 microliters, greater than or equal to 75 microliters, greater than or equal to 100 microliters, greater than or equal to 250 microliters, greater than or equal to 300 microliters, greater than or equal to 400 microliters, greater than or equal to 500 microliters, greater than or equal to 600 microliters, greater than or equal to 750 microliters, greater than or equal to 1 milliliter, greater than or equal to 1.3 milliliters, greater than or equal to 1.5 milliliters, greater than or equal to 2 milliliters, greater than or equal to 5 milliliters, greater than or equal to 10 milliliters, greater than or equal to 20 milliliters, greater than or equal to 30 millimeters, greater than or equal to 50 milliliters, greater than or equal to 70 milliliters, greater than or equal to 90 milliliters or greater than or equal to 100 milliliters. In some embodiments, the internal volume (e.g., holding volume) of the narrow tube may be less than or equal to 100 milliliters, less than or equal to 90 milliliters, less than or equal to 70 milliliters, less than or equal to 50 milliliters, less than or equal to 30 millimeters, less than or equal to 20 milliliters, less than or equal to 10 milliliters, less than or equal to 5 milliliters, less than or equal to 10 milliliters, less than or equal to 5 milliliters, less than or equal to 2 milliliters, less than or equal to 1.5 milliliters, less than or equal to 1.3 milliliters, less than or equal to 1 milliliter, less than or equal to 750 microliters, less than or equal to 600 microliters, less than or equal to 500 microliters, less than or equal to 400 microliters, less than or equal to 300 microliters, less than or equal to 250 microliters, less than or equal to 100 microliters, less than or equal to 75 microliters, less than or equal to 50 microliters, less than or equal to 30 microliters, less than or equal to 25 microliters, less than or equal to 20 microliters, less than or equal to 15 microliters, less than or equal to 12 microliters, less than or equal to 10 microliters, less than or equal to 5 microliters, less than or equal to 2 microliters, or less than or equal to 1 microliter. Combinations of the above-referenced ranges are also possible (e.g., greater than or equal to 1 microliter and less than or equal to 100 microliters). Other ranges are possible.

As described herein, in some embodiments, the narrow tube is connected to and/or within a barrel. In some embodiments, a ratio of an average diameter of the barrel to the average diameter of the narrow tube is greater than or equal to 1:1, 1.1:1, 1.2:1, 1.3:1, 1:5:1, 2:1, 2.5:1, 3:1, 4:1, 5:1, 10:1 or greater. In some embodiments, a ratio of an average diameter of the barrel to the average diameter of the narrow tube is less than or equal to 10:1, 5:1, 4:1, 3:1, 2.5:1, 2:1, 1.5:1, 1.3:1, 1.2:1, 1.1:1, or 1:1. Combinations of the foregoing range are possible (e.g., greater than or equal to 1:1 and less than or equal to 10:1). Other ranges are possible.

Some embodiments in accordance with the disclosed subject matter inexpensive to manufacture and easy to use.

Some embodiments in accordance with the disclosed subject matter may be disposable, single use devices. This promotes safety by avoiding contamination and avoids the need for cleaning and sanitation required with reusing devices. However, in other embodiments, the disclosed subject matter may be useable and may be configured to process more than one sample without disposing of the syringe.

Certain prior sample preparation methods are of moderate to high complexity and require testing at a CLIA-accredited facility. Certain embodiments in accordance with the disclosed subject matter do not expose the user to the sample, expanding use beyond CLIA-certified laboratories to potentially all point-of-care settings. Certain embodiments in accordance with the disclosed subject matter are simple to operate, as the workflow is not technique-dependent and requires no calibration. Certain embodiments in accordance with the disclosed subject matter therefore allow for use in point-of-care settings, enabling faster results for patients and healthcare providers by reducing bottlenecks incurred by outsourcing diagnostic tests.

Certain embodiments in accordance with the disclosed subject matter allow for rapid testing as well, as the entire workflow can take only approximately a minute, or from just seconds to a few minutes, to transfer a sample to a reaction container. The simple operation means less training and time is required and reduces the possibility for errors.

Certain embodiments in accordance with the disclosed subject matter facilitate safety. For example, a non-sharp pipette tip may be used (or, in other cases, a sharp tip may be used). In some embodiments, the tip is covered to reduce the risk of user exposure. Some embodiments reduce the risk of accidental dispensing of the fluid sample.

Persons of ordinary skill in the art will appreciate that the implementations of the invention encompassed by the disclosure are not limited to the particular examples described above. For example, while various embodiments have been described in the context of RT-PCR, it will be understood by persons of ordinary skill in the art that other applications are possible. For example, certain embodiments can be used for the preparation of a sample other than for RT-PCR, such as chromatography (e.g., liquid chromatography, gas chromatography). In some such embodiments, the reaction container may comprise chromatography reagents (e.g., ionization enhancers, ionization suppressors, internal standards). Although illustrative implementations have been shown and described, a wide range of modification, change, and substitution is contemplated in the foregoing disclosure. For example, features of the disclosed embodiments may be combined, rearranged, etc., within the scope of the invention to produce more embodiments. It is understood that such variations may be made to the foregoing without departing from the scope of the disclosure. Accordingly, it is appropriate that the appended claims be construed broadly, consistent with the disclosure.

1. A syringe assembly for transferring a fluid sample for testing, the syringe assembly comprising:
a housing comprising a side wall, a proximal end, and a distal end;
a barrel comprising an interior, a proximal end, and a distal end, wherein at least the distal end of the barrel is located inside the housing;
a plunger reciprocally moveable in the interior of the barrel in a proximal direction and in a distal direction;
a narrow tube having an interior, wherein a proximal end of the interior of the narrow tube is in fluid communication with a distal end of the interior of the barrel, wherein a distal tip of the narrow tube has an opening, and wherein the plunger, barrel, and narrow tube define a fluid chamber within the syringe assembly; and
a metering actuator configured to control movement of the plunger at least in a proximal direction relative to the barrel;
wherein the barrel, the plunger, and the narrow tube are configured such that relative movement between the plunger and the barrel expanding the fluid chamber within the syringe assembly causes a fluid sample to be drawn through the opening of the narrow tube into the fluid chamber within the syringe assembly;
wherein the housing is configured to receive a reaction container at the distal end of the housing;
wherein the fluid chamber is configured to hold the fluid sample after the fluid sample has been drawn into the fluid chamber and before the fluid sample is expelled from the fluid chamber into the reaction container;
wherein the syringe assembly is configured such that while the fluid chamber holds the fluid sample, the distal tip of the narrow tube remains covered; and
wherein the barrel, the plunger, and the narrow tube are configured such that relative movement between the plunger and the barrel contracting the fluid chamber causes the fluid sample to be expelled from the fluid chamber through the opening of the narrow tube into the reaction container.

2. The syringe assembly as recited in claim 1, wherein the metering actuator comprises a stem and a cap, wherein the stem is connected to the plunger, wherein the cap comprises a slot for receiving the stem, wherein the slot has a first stop and a second stop, wherein the syringe assembly is configured to draw a fluid sample into the fluid chamber by movement of the stem from the first stop to the second stop, and wherein the syringe assembly is configured to expel the fluid sample from the fluid chamber into the reaction container by movement of the stem from the second stop to the first stop.

3. The syringe assembly as recited in claim 1, wherein the syringe assembly further comprises a sample chamber inside the housing and a pierceable cover at a bottom of the sample chamber, wherein the syringe assembly is configured to draw the fluid sample from the sample chamber into the fluid chamber by actuation of the plunger in the proximal direction, and wherein the barrel and narrow tube are configured to be advanced relative to the housing after the fluid sample has been drawn into the fluid chamber such that the narrow tube pierces the cover at the bottom of the sample chamber in order to expel the fluid sample from the fluid chamber into the reaction container.

4. The syringe assembly as recited in claim 1, wherein the metering actuator comprises a cap, a first threaded surface rotatable with the cap, and a second threaded surface, wherein the first threaded surface is in threaded engagement with the second threaded surface, wherein the cap and the first threaded surface are rotatable with respect to the housing, and wherein rotation of the cap with respect to the housing is configured to cause longitudinal movement of the plunger with respect to the barrel due to the engagement of the first threaded surface with the second threaded surface.

5. The syringe assembly as recited in claim 1, wherein the metering actuator comprises a knob, at least one projection moveable with the plunger, and at least one opening in the housing, and wherein the at least one projection is configured to engage the opening to control the amount of movement of the knob.

6. The syringe assembly as recited in claim 1, wherein the barrel is configured to be moved proximally against a spring to cause the relative movement between the plunger and the barrel contracting the fluid chamber and expelling the fluid sample from the fluid chamber through the opening of the narrow tube into the reaction container.

7. A set of components usable together for transferring a fluid sample for testing, the set of components comprising:
a reaction container assembly comprising a reaction container having an opening and a pierceable cover covering the opening of the reaction container; and
a syringe assembly, the syringe assembly comprising:
a housing comprising a side wall, a proximal end, and a distal end;
a barrel comprising an interior, a proximal end, and a distal end, wherein at least the distal end of the barrel is located inside the housing;
a plunger reciprocally moveable in the interior of the barrel in a proximal direction and in a distal direction;
a narrow tube having an interior, wherein a proximal end of the interior of the narrow tube is in fluid communication with the interior of the barrel, wherein a distal tip of the narrow tube has an opening, and wherein the plunger, barrel, and narrow tube define a fluid chamber within the syringe assembly; and
a metering actuator configured to control movement of the plunger at least in a proximal direction relative to the barrel;
wherein the barrel, the plunger, and the narrow tube are configured such that relative movement between the plunger and the barrel expanding the fluid chamber within the syringe assembly causes a fluid sample to be drawn through the opening of the narrow tube into the fluid chamber within the syringe assembly;
wherein the housing is configured to receive the reaction container assembly at the distal end of the housing;
wherein the fluid chamber is configured to hold the fluid sample after the fluid sample has been drawn into the fluid chamber and before the fluid sample is expelled from the fluid chamber into the reaction container;
wherein the syringe assembly is configured such that while the fluid chamber holds the fluid sample, the distal tip of the narrow tube remains covered; and
wherein the barrel, the plunger, and the narrow tube are configured such that relative movement between the plunger and the barrel contracting the fluid chamber causes the fluid sample to be expelled from the fluid chamber through the opening of the narrow tube into the reaction container.

8. The syringe assembly as recited in claim 7, wherein the distal tip of the narrow tube is configured to remain at all times located inside the housing proximal to the distal end of the housing or located inside the reaction container.

9. The set of components as recited in claim 7, wherein the set of components further comprises a collection container assembly comprising a collection container having an opening and a pierceable cover covering the opening of the collection container, wherein the housing is configured to receive the collection container assembly at the distal end of the housing, and wherein the syringe assembly is configured to draw the fluid sample from the collection container into the fluid chamber by actuation of the plunger in the proximal direction.

10. The set of components as recited in claim 9, further comprising a collection container stop for positioning the collection container with respect to the syringe assembly.

11. The set of components as recited in claim 7, wherein the reaction container assembly further comprises reaction container holder, wherein the side wall of the housing has a track, and wherein the reaction container holder has a projection that fits in the track.

12. The set of components as recited in claim 7, further comprising a proximal reaction container stop for positioning the reaction container in a proximal position with respect to the housing for expelling the fluid sample into the reaction container and a distal reaction container stop for positioning the reaction container in a distal position with respect to the housing for exposing the reaction container for external processing.

13. The set of components as recited in claim 7, wherein the metering actuator comprises a knob, at least one projection moveable with the plunger, and at least one opening in the housing, and wherein the at least one projection is configured to engage the opening to control the amount of movement of the knob.

14. The set of components as recited in claim 7, wherein the barrel is configured to be moved proximally against a spring to cause the relative movement between the plunger and the barrel contracting the fluid chamber and expelling the fluid sample from the fluid chamber through the opening of the narrow tube into the reaction container.

15. The set of components as recited in claim 7, wherein the metering actuator comprises a cap, a first threaded surface rotatable with the cap, and a second threaded surface, wherein the first threaded surface is in threaded engagement with the second threaded surface, wherein the cap and the first threaded surface are rotatable with respect to the housing, and wherein rotation of the cap with respect to the housing is configured to cause longitudinal movement of the plunger with respect to the barrel due to the engagement of the first threaded surface with the second threaded surface.

16. A method of transferring a fluid sample for testing, the method comprising:
(i) using a syringe assembly to draw a fluid sample, the syringe assembly comprising:
a housing comprising a side wall, a proximal end, and a distal end;
a barrel comprising an interior, a proximal end, and a distal end, wherein at least the distal end of the barrel is located inside the housing;
a plunger reciprocally moveable in the interior of the barrel in a proximal direction and in a distal direction;
a narrow tube having an interior, wherein a proximal end of the interior of the narrow tube is in fluid communication with a distal end of the interior of the barrel, wherein a distal tip of the narrow tube has an opening, and wherein the plunger, barrel, and narrow tube define a fluid chamber within the syringe assembly; and
a metering actuator configured to control movement of the plunger at least in a proximal direction relative to the barrel;
wherein using the syringe assembly to draw the fluid sample comprises creating relative movement between the plunger and the barrel expanding the fluid chamber within the syringe assembly thereby causing the fluid sample to be drawn through the opening of the narrow tube into the fluid chamber within the syringe assembly;
(ii) causing a reaction container to be received at the distal end of the housing;
(iii) holding the fluid sample in the fluid chamber after the fluid sample has been drawn into the fluid chamber and before the fluid sample is expelled from the fluid chamber into the reaction container, wherein while the fluid chamber is holding the fluid sample, the distal tip of the narrow tube remains covered; and
(iv) creating relative movement between the plunger and the barrel contracting the fluid chamber thereby causing the fluid sample to be expelled from the fluid chamber through the opening of the narrow tube into the reaction container.

17. The method as recited in claim 16, wherein using the syringe assembly to draw the fluid sample is performed while the distal tip of the narrow tube is located inside the housing proximal to the distal end of the housing.

18. The method as recited in claim 16, wherein the distal tip of the narrow tube is configured to remain at all times located inside the housing proximal to the distal end of the housing or located inside the reaction container.

19.-21. (canceled)

22. The method as recited in claim 16, wherein using the syringe assembly to expel the fluid sample into the reaction container comprises moving the barrel proximally against a spring to cause the relative movement between the plunger and the barrel contracting the fluid chamber and expelling the fluid sample from the fluid chamber through the opening of the narrow tube into the reaction container.

23. (canceled)

24. The method as recited in claim 16, wherein the reaction container is a polymerase chain reaction container, and further comprising, after expelling the fluid sample from the barrel into the reaction container, exposing the reaction container to one or more polymerase chain reaction processing steps while the reaction container remains secured to the syringe assembly.
